(19) United States
(12) Patent Application Publication
(10) Pub. No.: US 2011/0207820 A1
(43) Pub. Date: Aug. 25, 2011

(54) NOVEL CHRYSOCHROMULINA SPECIES, METHODS AND MEDIA THEREFOR, AND PRODUCTS DERIVED THEREFROM

(75) Inventors: Rose Anne Cattolico, Seattle, WA (US); Mark S. Cooper, Seattle, WA (US); William Robert Hardin, New Castle, WA (US); Nicholas Bigelow, Seattle, WA (US); Cong-Thien Vo, Seattle, WA (US); Chloe R. Deodato, Seattle, WA (US)

(73) Assignee: UNIVERSITY OF WASHINGTON, Seattle, WA (US)

(21) Appl. No.: 12/887,415

(22) Filed: Sep. 21, 2010

Related U.S. Application Data
(63) Continuation-in-part of application No. PCT/US2009/037997, filed on Mar. 23, 2009.
(60) Provisional application No. 61/245,225, filed on Sep. 23, 2009.
(60) Provisional application No. 61/038,428, filed on Mar. 21, 2008.

(51) Int. Cl. A61K 31/20; C12Q 1/02; C12N 1/12; C10L 1/188; A23D 9/00

(52) U.S. Cl. 514/560; 435/29; 435/257.1; 44/385; 426/601

(57) ABSTRACT

An algal cell that is capable of surviving in fresh water and has a high fatty acid content, an algal culture including the algal cell, methods of growing the algal culture, algal growth media, and methods for selectively adapting the algal culture are provided.

### CROSS-REFERENCES TO RELATED APPLICATIONS

This application claims the benefit of Provisional Application No. 61/245,225, filed Sep. 23, 2009, and is a continuation-in-part of International Application No. PCT/US2009/037997, filed Mar. 23, 2009, which claims the benefit of Provisional Application No. 61/038,428, filed Mar. 21, 2008, the disclosures of which are incorporated herein by reference in their entirety.

### BACKGROUND

The impact of greenhouse gas emissions on global warming, the rapidly increasing demand for limited oil reserves, and the political instability of the Middle East are now driving the development of alternatives to petrochemicals. Biodiesel fuel is derived from living organisms, and produced by the transesterification of fatty acids to alkyl esters. With only small adjustments in technology, biodiesel can be made available for use in transportation, heating, and industrial uses. Historically, biostock for biofuel has been obtained from terrestrial plants such as soy, sunflower, or palm. Although plant oil yields range from about 20 to 100 gallons/acre/year, these sources often negatively impact both the production of food crops and the integrity of fragile ecosystems.

Algae provide an attractive alternative source of biostock for diesel production. These organisms produce significantly higher volumes of fatty acids per acre than conventional crop plants. Moreover, unlike crop plants, algae can be cultured on land unsuited for crops and harvested 365 days a year. Moreover, algae-derived biodiesel fuel is eco-friendly, renewable, biodegradable, and non-toxic. In addition to biofuel production, such algae may also be an important source of fatty acids for pharmaceuticals, nutraceuticals, cosmetics, foods, dietary supplements, as well as for a variety of other uses.

The technological challenges in establishing algae programs are multifaceted. While some wild-type algae are suitable for use in biofuel and other applications, algal strain modifications (for example, by selection or directed evolution) of wild-type algae to improve particular characteristics useful for biofuel and other applications are more likely to be commercially viable. Among the most critical aspects of algal biostock development are the identification of algae that are efficient fatty acid producers, and the scale-up of such algae for commercial production.

Algae oil harvest enhancement opportunities include the following: (1) isolating new strains of algae that produce high amounts of desirable oils; (2) identifying growth conditions to promote rapid growth of the oil-producing algae; (3) identifying life cycle behaviors of algae that can be used to optimize the commercial harvesting of algae oil crops. Hence, there exists a need for improved algal biostocks suitable biofuel and other applications, as well as improved methods and media for adapting and growing such algal bio stocks.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a method of growing an algal culture having a fatty acid content of at least 5.0×10̂-12 grams is provided. The method generally includes adding an algal culture to a growth medium including water, an alkaline buffer solution, a trace metal ion solution, a vitamin solution, phosphate, and nitrogen. The method further includes exposing the algal culture to a light condition greater than about 60 μE/m²/sec, wherein the light schedule includes at least 6 hours of light followed by at least 6 hours of darkness.

In accordance with another embodiment of the present disclosure, a method of selectively generating an algal culture having an identification property is provided. The method includes obtaining a first algal culture having an identification property having a first value, isolating the first algal culture in a first growth medium, incubating the first algal culture in the first growth medium to provide a second algal culture, and sorting the second algal culture to select algal cells having the identification property having a second value to provide a sorted portion of the second algal culture.

In accordance with another embodiment of the present disclosure, a fatty acid mixture obtained from an alga is provided. The fatty acid mixture includes C14 in an amount in the range of about 14 to about 25 weight percent of the total lipid content, C16 in an amount in the range of about 17 to about 26 weight percent of the total lipid content, C18 in an amount in the range of about 29 to about 57 weight percent of the total lipid content, and C20 and greater in an amount in the range of about 9 to about 30 weight percent of the total lipid content.

In accordance with another embodiment of the present disclosure, an algal cell is provided. The algal cell has an average fatty acid content of at least about 5.0×10̂-12 grams, and the algal cell is capable of surviving in fresh water.

In accordance with another embodiment of the present disclosure, an algal culture is provided. The algal culture generally includes a plurality of algal cells having an average fatty acid content of at least 5.0×10̂-12 grams per cell, and the algal culture is capable of surviving in fresh water.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an electron micrograph of an alga organism (Alga X) at about 6500× magnification, in accordance with embodiments of the present disclosure;

FIG. 2 is a relatedness tree for Alga X based on sequencing information from 18S ribosomal RNA;

FIG. 3 is a comparative growth curve for Alga X in various media, in accordance with embodiments of the present disclosure;

FIG. 4 is a growth curve showing the cell growth (cell concentration in cells/ml and lipid content in lipid/cell) in response to light cycle for Alga X over a 12 hour time period, in accordance with embodiments of the present disclosure;

FIG. 5 is a comparative plot of Alga X growth as a result of changing pH buffer and nutrition;

FIG. 6 is a comparative plot of lipid content (fatty acid/cell) versus cell concentration (cells/ml) for Alga X in various media, in accordance with embodiments of the present disclosure;

FIG. 7 is a semi-continuous batch culture growth curve showing cell growth in response to harvest patterns, in accordance with embodiments of the present disclosure;

FIG. 8 is a fluorescent micrograph of an Alga X cell at about 100× magnification having lipid bodies dyed with BODIPY 505/515, in accordance with embodiments of the present disclosure;

FIG. 9 is a long-term semi-continuous batch culture growth curve showing cell growth in response to harvest patterns, in accordance with embodiments of the present disclosure; and

FIG. 10 is a comparative plot of lipid content (fatty acid/cell) versus cell concentration (cells/ml) for Alga X after applying directed evolution techniques.

### DETAILED DESCRIPTION

Embodiments of the present disclosure are directed to algal organisms, cultures, methods and media for adaptation and growth, and products derived from the alga cultures, as described in greater detail below. Although the algal organism described herein is directed to an alga species, it should be appreciated that the methods and media for adaptation and growth, and the products derived from the alga cultures are not limited to such algal species. As non-limiting examples, organisms from the following algal classes will grow in the media or a modification of the media and in accordance with the methods described herein: Chrysochromulina (Haptophyceae; naturally grows in freshwater); Chlamydomonus (Chlorophyceae; naturally grows in freshwater); Nannochloropsis (Eustigamatophyceae; naturally grows in brackish water); Chomulina (Chrysophyceae; naturally grows in brackish water); Synura (Synurophyceae; naturally grows in fresh water), and Oscilliatoria (Cyanophyceae; naturally grows in brackish water).

### Organism and Culture

The alga organism is a new species, which has been isolated and cultivated as a pure algal culture, is believed to belong to the Class of Chrysochromulina, Order of sp. (unknown species).

A quantity of commercially available parental Chrysochromulina sp. (hereinafter “C. sp.”) was cultivated under atypical conditions (for example, including but not limited to high temperature, high pH, low light, salinity, and media changes), and also by using a selection process for directed evolution of high fatty acid generation cells, as described in greater detail below. These cultivation conditions created strain variants (hereinafter collectively referred to as “Alga X”) rendered apparent because of, for example, a significantly increased lipid (or fatty acid) content and a significantly increased growth rate, when compared to lipid content and growth rate of the parental strain.

The parental (or “wild type”) strain is available from, for example, the Provasoli-Guillard National Center of Marine Phytoplankton (CCMP) (Boothbay, Me., USA) Collection of Algae, Collection Number CCMP.291. The parental strain was collected from approximately 39.0000N 105.0000W Colorado, USA. In nature, the parental strain is small in size (generally about 5 to about 6 microns), fragile, unicellular, and prefers cool temperatures, such as about 11° C. to about 16° C. (about 52° F. to about 61° F.), and grows in population at a slow rate (for example, dividing about once every four days). Microscopic analysis shows that each algal cell contains at least one large lipid body, and generally between one and three large lipid bodies.

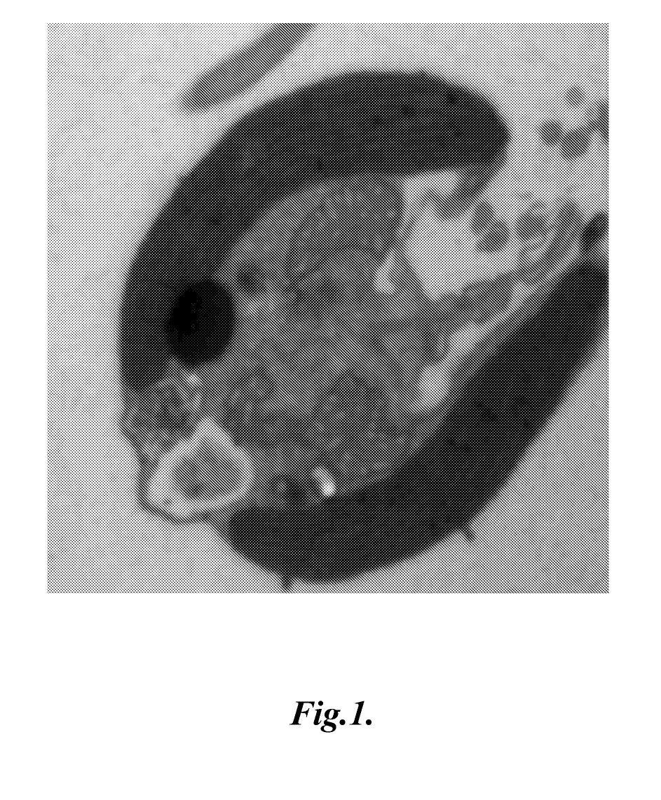

Alga X, as selected for and cultured, now fulfills several criteria that make it attractive as a potential biofuel source, including but not limited to high lipid content (see large black lipid body in FIG. 1), rapid growth rate, ability to grow to high density in a culture, ability to grow on waste water, low light requirements in the growth medium, ambient temperature growth, growth in fresh water or low salinity medium, non-biofouling growth, and being delineated solely by a plasma membrane (see FIG. 1), as described in greater detail below. It should be appreciated that Alga X may be cultured as a non-axenic, unialga culture.

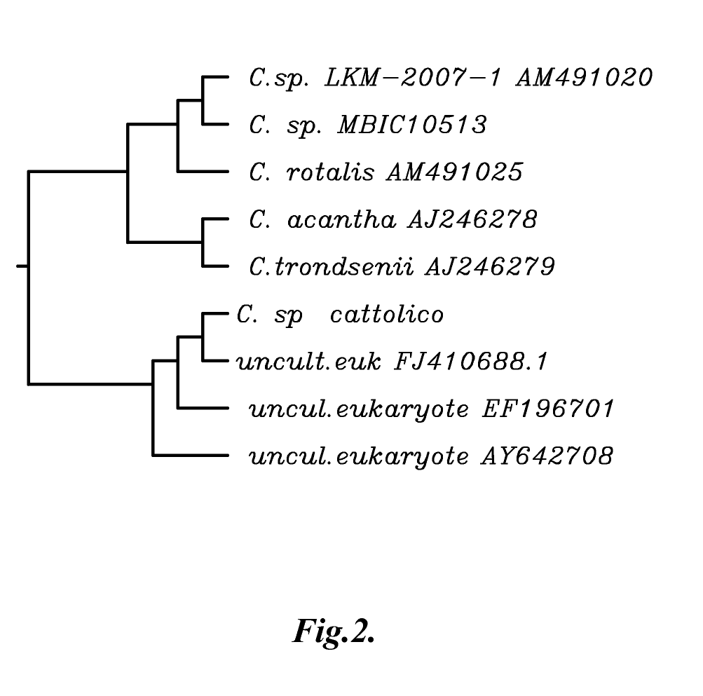

A partial 18S ribosomal RNA sequence shows Alga X to be sister to the Chrysochromulina assemblage, but separate from described Chrysochromulina species (see FIG. 2). Additionally, as seen in the Clustal-W alignment below, Algal X strain differs in the partial 18S ribosomal sequence from the parent culture (CCMP 291) by at least one nucleotide:

uncult.euk_FJ410688.1
GCTCGAATCGCATG-CTTCACGCCGGCGATGGTTCATTCAAATTTCTGCC
c.parvaAM491019 (from CCMP 291)
GCTCGAATCGCATG-CTTCACGCCGGCGATGGTTCATTCAAATTTCTGCC
uncul.eukaryoteEF196701
GCTCGAATCGCATGGCTTCACGCCGGCGATGGTTCATTCAAATTTCTGCC
C._sp._cattolico
GCTCGAATCGCATGGCTTCACGCCGGCGATGGTTCATTCAAATTTCTGCC
uncul.eukaryoteAY642708
GCTCGAATCGCATGGCTTCACGCCGGCGATGGTTCATTCAAATTTCTGCC
C._rotalis_AM491025
GCTCGAATCGCATGGCTTTACGCCGGCGATGGTTCATTCAAATTTCTGCC
C._sp._MBIC10513
GCTCGAATCGCATGGCTTTACGCCGGCGATGGTTCATTCAAATTTCTGCC
C.sp._LKM-2007-
GCTCGAATCGCATGGCTTTACGCCGGCGATGGTTCATTCAAATTTCTGCC
C._acantha_AJ246278
GCTCGAATCGCATGGCTTTACGCTGGCGATGGTTCATTCAAATTTCTGCC
C.trondsenii_AJ246279
GCTCGAATCGCATGGCTTTACGCTGGCGATGGTTCATTCAAATTTCTGCC.

On a dry weight basis, when measured by a Bligh-Dyer solvent extraction process for lipids, Alga X has a lipid content of 350 mg lipids per gram dry weight, which is significantly higher than comparative values for other common high lipid content algae, such as Chaetoceros, Chroomonas, Cyclotella, Duniella, Isochrysis, Nannochloropsis, Nannochloris, Phaeodactylum, Pavlovia, and Tetraselmis, as seen in EXAMPLE 1 below. Methods and mediums for increasing the lipid content in Alga X are described in greater detail below.

In one embodiment of the present disclosure, Alga X has a lipid content of at least 250 mg lipids per gram dry weight, when measured by a solvent extraction process for lipids. In another embodiment of the present disclosure, Alga X has a lipid content of at least 300 mg lipids per gram dry weight, when measured by a solvent extraction process for lipids. In another embodiment of the present disclosure, Alga X has a lipid content of at least 350 mg lipids per gram dry weight, when measured by a solvent extraction process for lipids. In another embodiment of the present disclosure, Alga X has a lipid content of at least 400 mg lipids per gram dry weight, when measured by a solvent extraction process for lipids. In another embodiment of the present disclosure, Alga X has a lipid content of at least 450 mg lipids per gram dry weight, when measured by a solvent extraction process for lipids. n another embodiment of the present disclosure, Alga X has a lipid content of at least 550 mg lipids per gram dry weight, when measured by a solvent extraction process for lipids.

In addition to high lipid content, the lipid profile of Alga X is also of high value. It should be appreciated that total lipid content includes fatty acids as well as other lipid types. Fatty acids may include saturated fatty acids and unsaturated fatty acids. The oil in Alga X includes, but is not limited to, the following fatty acids (including shorthand name, systematic name, common name, and/or any other well-known name):

C14:0 (Tetradecanoic Acid; Myristic Acid) (saturated);

C16:0 (Hexadecanoic Acid; Palmitic Acid) (saturated);

C16:1 omega-7 (cis-9-Hexadecenoic Acid; Palmitoleic Acid) (unsaturated);

C18:0 (Octadecanoic Acid; Stearic Acid) (saturated);

C18:1 omega-7 (cis-11-Octadecenoic Acid; cis-Vaccenic Acid) (unsaturated);

C18:1 omega-9 (cis-9-Octadecenoic Acid; Oleic Acid) (unsaturated);

C18:2 omega-6 (cis-9-cis-12-Octadecadienoic Acid; Linoleic Acid) (unsaturated);

C18:3 omega-3 (cis-9-cis-12-cis-15-Octadecatrienoic Acid; alpha-Linolenic Acid; ALA) (unsaturated);

C18:3 omega-6 (cis-6-cis-9-cis-12-Octadecatrienoic Acid; gamma-Linolenic Acid; GLA) (unsaturated);

C20:4 omega-6 (cis-5-cis-8-cis-11-cis-14-Eicosatetraenoic Acid; Arachidonic Acid) (unsaturated);

C20:5 omega-3 (cis-5-cis-8-cis-11-cis-14-cis-17-Eicosapentaenoic Acid; Timnodonic Acid; EPA) (unsaturated);

C22:5 omega-3 (cis-7-cis-10-cis-13-cis-16-cis-19-Docosapentaenoic Acid; Clupanodonic Acid) (unsaturated); and

C22:6 omega-3 (cis-4-cis-7-cis-10-cis-13-cis-16-cis-19-Docosahexaenoic Acid; Cervonic Acid; DHA) (unsaturated).

Individual Alga X cells have an average fatty acid content of greater than 2.0×10̂-12 grams per cell measured by gas chromatography mass spectroscopy (GC/MS). In another embodiment, individual Alga X cells have an average fatty acid content of at least about 5.0×10̂-12 grams per cell. In another embodiment, individual Alga X cells have an average fatty acid content of at least about 10.0×10̂-12 grams per cell. In another embodiment, individual Alga X cells have an average fatty acid content of at least about 5.0×10̂-12 grams per cell to about 20.0×10̂-12 grams per cell. This fatty acid content compares to the parental alga cell having on average about 2.0×10̂-12 grams per cell.

Optimization of fatty acid content (amount and fatty acid type), along with growth rate, is important when developing an alga for commercialization. There was no previous fatty acid analysis reported for the parental alga before such data was collected for the present disclosure. However, data below in EXAMPLE 29 presents total fatty acid content and fatty acid identities obtained by GC/MS analysis of Alga X cultures in different media. Notably, the parental strain is maintained in DYV at the CCMP Collection.

As mentioned above, the initial estimate of fatty acid content for the parental alga before applying selective pressure was about 2.0×10⁻¹² g/cell. This value is higher than those reported for other algal species, such as Dunaliella salina (Weldy & Husemann, Lipid Production by Duniella salina in Batch Culture: Effects of Nitrogen Limitation & Light Intensity, U.S. Dept. Energy J. Undergraduate Research, 115-22, on-line through the Dept. of Energy Office of Science); Isochrysis galbana (EP 19910304789); Crypthecodinium cohnii (U.S. Pat. No. 5,711,983); or numerous other algae (Brown, Amino Acid and Sugar Composition of Sixteen Species of Microalgae Used in Mariculture, 145 Aquaculture, 79-99 (1991).

Importantly, gas chromatographic analysis results show that unsaturated fatty acids constitute a large proportion of the recovered lipids in the parental strain. Many are omega-3 or omega-6 unsaturated fatty acids. One of these, C18:5n3, is uncommon outside of algal sources. The importance of omega-3 and omega-6 fatty acids is extensively documented. Though normally obtained from fish oil (sometimes contaminated with pollutants), this product is used for a broad range of pharmaceutical applications.

Achieving a high percentage of saturated fatty acids compared to unsaturated fatty acids in a shorter lifespan is important for commercialization of biofuels, because more biostock output can be achieved in a shorter period of time using the same equipment for culturing the algae. Therefore, when optimized in accordance with the methods described herein, Alga X has a high percentage of saturated fatty acids in the range of about 20% to about 70%, as compared to other common high lipid content algae. For example, see EXAMPLE 2 below (69% by weight saturated fatty acids; 31% by weight unsaturated fatty acids). In another embodiment, Alga X has a high percentage of saturated fatty acids in the range of about 35% to about 55%. As discussed in greater detail below, there is significant variability in the fatty acid composition of the Alga X cells based on several factors in the growth cycle, including but not limited to light intensity, light cycle, temperature, nutrients, salinity, pH, water source, and harvest rate.

In one embodiment of the present disclosure, the specific fatty acid distribution in the cells includes the following: (a) C14 in an amount in the range of about 14 to about 25 weight percent of the total lipid content; (b) C16 in an amount in the range of about 17 to about 26 weight percent of the total lipid content; (c) C18 in an amount in the range of about 29 to about 57 weight percent of the total lipid content; and (d) C20 and greater in an amount in the range of about 9 to about 30 weight percent of the total lipid content. “C20 and greater” generally includes C20 and may include amounts of C22 and C24.

In addition to a high lipid content and desirable fatty acids, Alga X cells grow rapidly to high density in an algal culture. As mentioned above, the parental alga strain, as obtained initially from the culture collection, divides about once every four days. In contrast, the Alga X culture selected for and described herein divides at least once per day, but can divide at least twice per day, or at least three times per day, or in the range of one to three times per day, a significant change from the parental alga strain. In accordance with embodiments of the present disclosure, the algal culture density may be in the range of about 1×10̂6 to about 1×10̂8.

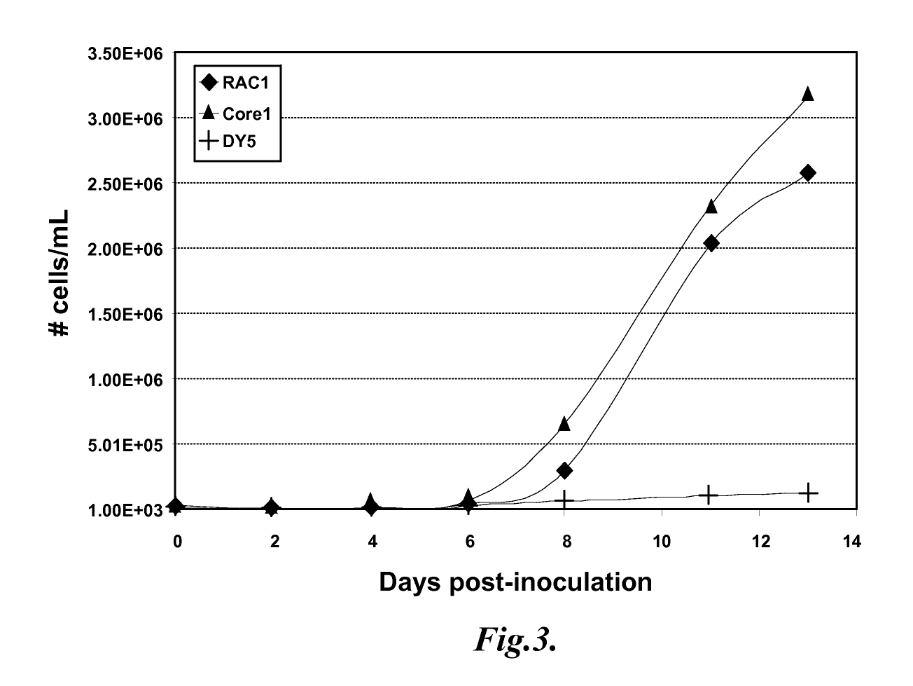

The maximum density of Alga X cultures achieved thus far under laboratory-controlled conditions is typically about 7.2×10̂6 cells/ml. FIG. 3 represents a typical growth curve for Alga X in various different mediums, which shows that the culture medium, as described in greater detail below, plays a significant role in culture densities achieved. Moreover, the inventors have found that they can grow an alga culture originating from a single Alga X cell.

Not only can high culture density be achieved, but Alga X cells are also able to grow to high density in large batch cultures. Often, algae can be grown to high cell density in small laboratory cultures, but then fail to thrive when even moderately-sized culture volumes are attempted. This issue presents a problem when choosing an alga that will be useful in mass culture. Different Alga X cultures have been grown in several large batches. For example, using the RAC1 medium (see EXAMPLE 13 below for a RAC1 medium recipe) growth of Alga X in a 1240 liter tank to a cell density of 1.2×10⁶ cells/ml was achieved. Further, in a 5.5 liter flat tub using waster water medium (CORE1, SEM, and wastewater), a culture density of 6×10̂6 cells/ml was achieved in five days.

The mean size of Alga X is less than about 5 microns in diameter, or in some cases less than 4.5 microns in diameter, which is a little smaller than the cell size of the parental strain. This small size results in a large surface-to-volume ratio that is advantageous in large-batch culturing. Studies of numerous algal species show that harvestable cell mass is inversely proportional to cell volume. See Nielson, 28 J. Phytoplankton Res. 489-98 (2006). It should be appreciated that the growth of larger algal cells is often inhibited as self-shading increases with culture density.

Regarding light requirements, Alga X cells require extremely low light for growth as compared to the parental alga, which is generally grown on higher light intensity from natural sunlight, on the order of 2000+μE/m²/sec. The need for less light is a valuable characteristic because lighting “costs” with respect to energy input can be an important factor in commercialization feasibility. Experimental results for Alga X growth under various light intensity conditions are discussed in EXAMPLE 3 below.

In one embodiment, cell growth of Alga X can be obtained with a light intensity greater than about 3μE/m²/sec. In another embodiment, cell growth can be obtained with a light intensity in the range of about 3μE/m²/sec to about 160μE/m²/sec. In yet another embodiment, excellent growth can be obtained with a light intensity in the range of about 60μE/m²/sec to about 100μE/m²/sec. It should be appreciated that the light may be from a full spectrum light source or from cool white bulbs. By comparison, Nannochloropsis, another alga targeted for biofuel production, requires much higher light intensity.

Experimental results for Alga X growth under light/dark cycles are discussed in EXAMPLE 4 below. In a preferred growth method, Alga X cells are maintained on a 12 hour light (12 L), followed by a 12 hour dark cycle (12 D). In accordance with other growth methods, the light schedule may be selected from the following group: at least 12 hours of light (12 L); at least 12 hours of dark (12 D); at least 10 hours of light (10 L); at least 10 hours of dark (10 D); at least 8 hours of light (8 L); at least 8 hours of dark (8 D); at least 6 hours of light (6 L); and at least 6 hours of dark (6 D). Algal X had poor cell growth under continuous light.

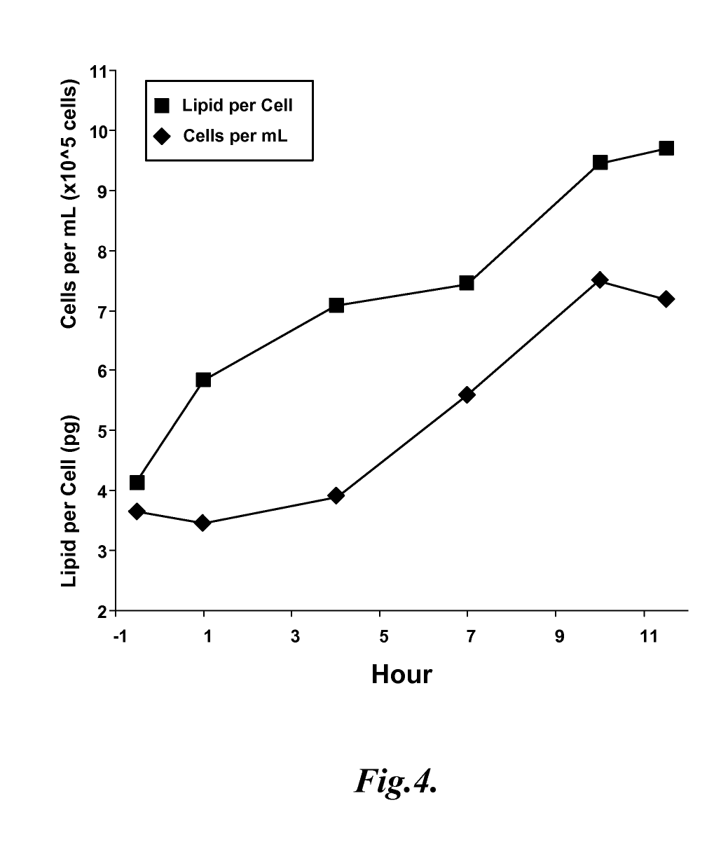

The inventors have discovered that Alga X operates according to the light/dark cell cycle for producing fatty acids, as seen in FIG. 4. See also EXAMPLE 5 below. In that regard, Alga X cells tend to increase in lipid content (lipids per cell) as they are exposed to light, reaching a maximum lipid content after several hours of light exposure. Accordingly, the inventors have found that Alga X cells may be harvested when they contain maximum lipid content. In accordance with one embodiment of the present disclosure, Alga X cells should be harvested after 6 hours of light exposure. In accordance with another embodiment of the present disclosure, Alga X cells should be harvested after 8 hours of light exposure. In accordance with another embodiment of the present disclosure, Alga X cells should be harvested after 10 hours of light exposure.

Moreover, the inventors have discovered that Alga X operates according to the light/dark cell cycle for cell division, as can be seen in FIG. 4. See also EXAMPLE 6 below. In that regard, Alga X cells tend to divide after several hours of light exposure. Therefore, an optimal harvest time can be determined based on optimizing the lipid content and cell division curves.

Regarding medium temperature, the parental alga, as obtained initially from the culture collection, had a suggested growth range in a temperature ranging from about 11° C. to about 16° C. (about 52° F. to about 61° F.). The improved Alga X culture described herein has been selected to display rapid growth at a higher temperature of about 20° C. (about 68° F.), as described in EXAMPLE 7 below. Moreover, selected alga cells can survive at about 24° C. (about 75° F.). Of note, in one experiment using a 16 hour light, 8 hour dark photoperiod, the growth of Alga X at about 18° C. (about 64° F.) was about 86% of the growth at 22.5° C. (about 73° F.). This experiment indicates that an Alga X culture can be successfully grown in cooler regions without requiring additional heating.

In one embodiment of the present disclosure, an Alga X culture temperature can be suitably maintained in the range of about 4 to about 24° C. (about 39° F. to about 75° F.). In another embodiment, the culture temperature can be suitably maintained in the range of about 4° C. to about 30° C. (about 39° F. to about 86° F.). In another embodiment, the culture temperature can be suitably maintained at greater than about 16° C. (about 61° F.). In yet another embodiment, the culture temperature can be suitably maintained at greater than about 20° C. (about 68° F.). The selection for cells that are temperature-tolerant and grow at an ambient temperature provides an advantage for scale up in that the range of the organism has been expanded, and cooling or heating energy is not required.

Regarding medium type, the parental alga is a fresh water organism. Therefore, Alga X is capable of growing in fresh water. Many of the current algae targeted for biofuel recovery are marine; thus, maintenance of these algae require large amounts of salts to generate artificial seawater growth medium, or transport of seawater to an algal growth facility. Notably, in these seawater media, nutrient additives are still required. Additionally, bioreactor components are subjected to extended saltwater exposure that compromises structural integrity. Moreover, growth of marine algae in coastal ocean facilities risks the introduction of exotic organisms into the local ecosystem. Alga X avoids many of these problems because of its fresh water growth abilities.

Although the parental alga cell naturally exists in fresh water, Alga X has been shown to grow in low salinity, brackish water, which allows for flexibility in growth media. Moreover, a low salt concentration in the medium has been shown to change the fatty acid composition of the Alga X cells, as described in greater detail below.

Regarding nutrient additives, Alga X cells are mixotrophic, being able to use both inorganic and organic carbon sources. This attribute allows a broader selection of media choices for its culture. Suitable nutrient additives include growth supplements, such as ALGA-GRO® Concentrated Medium (Carolina Biological Supply Company, Burlington, N.C.), soil extracts, and waster water, all discussed in greater detail below. Other additives studied include phosphate, nitrogen, acetate, metals (in the form of metal ions), vitamins, etc., all discussed in greater detail below.

Notably, the Alga X cells described herein are non-biofouling, i.e., they do not adhere to the walls of the culture vessel or tank, nor do they stick to one another, a property important for light transmission and harvesting. These cells neither clump nor foul their immediate environment, factors that affect the management of both cell culture and cell recovery. Moreover, Alga X cells can remain neutrally buoyant in fresh water, as a result of their size, high lipid content, and swimming capability. Therefore, no mechanical mixing is required to keep these cells uniformly distributed in the growth. In contrast, many non-flagellated cells or heavy-walled cells (e.g., diatoms and Chlamydomonas) sink unless subject to active agitation.

However, it should be appreciated that mixing Alga X cells at various mixing speeds is also within the scope of the present disclosure. As described below in EXAMPLE 32, Alga X cells in a culture subjected to agitation of about 60 rpm reached a desired cell concentration of 4.5E+06 in about 7 days. By comparison, alga X cells in a culture subjected to no agitation and about 30 rpm agitations reached the desired cell concentration in about 9 days.

Regarding oil retrieval, the Alga X cells are delineated solely by a plasma membrane, which means that there is essentially no outer cell wall, as can be seen in FIG. 1. No outer wall facilitates the retrieval of the valuable oils from the alga culture.

### Methods and Media for Growing Algal Cultures

An important goal of the commercial algal biofuel endeavor is to grow a large biomass of algae with a high lipid content at minimal cost. As discussed above, many algae targeted for biofuel recovery are marine. The maintenance of these marine organisms requires the use of large amounts of salts to generate an artificial seawater growth medium. Sometimes specialized salts are needed. For example, diatoms (frequently chosen as a biofuel source) require silica for wall development. If a natural seawater medium is chosen, enormous volumes of sea-water must be transported to the algal growth facility. Even with a seawater medium, salt and nutrient additives are often required to optimize the culture growth. An additional disadvantage of using marine algae as a biofuel source is that bioreactor components are subjected to extended saltwater exposure. Even stainless steel will eventually corrode in the presence of a saltwater medium.

It has been suggested that algae could be grown as a biostock in large coastal facilities. Unfortunately, coastal facilities may also present difficulties. For example, the “growth medium”, i.e. natural, in situ seawater may not be appropriate for the maintenance of high through-put, high lipid algal production. Additionally, the detrimental environmental effects of introducing “exotic” organisms to a coastal site must also be considered. The ability of Alga X to grow in a fresh water medium circumvents these potential problems.

One goal of the present disclosure provides for a low-cost medium that supports the generation of high cell densities in commercial, large batch algal cultures. Because the mean size of Alga X is less than about 5 microns, it has a large surface to volume ratio that is advantageous when considering growth potential. The CCMP maintains Alga X on DYV medium (available from Provasoli-Guillard National Center Culture Marine Phytoplankton, Bigelow Lab. Ocean Studies, West Bothbay, Me.). However, growth of Alga X under the conditions in the DYV medium is not optimized. A recipe for DYV medium having a pH 6.8 is included below in EXAMPLES 9-11.

Embodiments of the present disclosure are directed to newly devised growth media that allow the alga to divide rapidly and produce high lipid contents having desirable fatty acid compositions. These growth media have been optimized to reduce algal culture costs while maintaining cell division and lipid content efficiency. Factors for improving growth media may include but are not limited to salinity, buffering, supplemental nutrients, vitamins, macro-nutrients, and micro-nutrients, that may be considered when optimizing a medium for high density, high lipid algal culture having a specific fatty acid profile.

Notably, a number of media, for example, Bold's Basal Medium, Bischoff & Bold, 6318 Univ. Texas Pub. (1963), have failed to adequately support the growth of Alga X. In addition, conventional salt water media F/2 and L/1 made in fresh water also failed to adequately support the growth of Alga X. While not wishing to be bound by theory, it is believed that one factor in these failures may have been the starting pH and/or the buffering pH being too acidic.

As mentioned above, the growth media are not only for growing Alga X. For example, the following algal representatives will grow in the media and in accordance with the methods described herein: Chrysochromulina (naturally grows in freshwater); Chlamydomonus (naturally grows in freshwater); Nannochloropsis (naturally grows in brackish water); Chomulina (naturally grows in brackish water); Synura (naturally grows in freshwater); and Oscilliatoria (naturally grows in brackish water).

DYV generally includes the following ingredients: B-glycerolphosphate, phosphate (for example, added to the medium in the form KH₂PO₄), nitrogen (for example, added to the medium in the form of NO₃⁻ or NH₄⁺), acetate (NH₃CH₃COO⁻), magnesium (for example, added to the medium in the form of MgSO₄.7H₂O), potassium (for example, added to the medium in the form of KCl), boron (for example, added to the medium in the form of H₃BO₃), iron (for example, added to the medium in the form of FeCl₃.6H₂O) and a compound to keep the iron in solution, for example Na₂EDTA.2H₂O, calcium (for example, added to the medium in the form of CaCl₂), buffer solution, a trace metal ion solution (for example, including but not limited to manganese, zinc, cobalt, molybdenum, and vanadium metal ions and selenium ions, see EXAMPLE 12), and a vitamin solution (for example, including but not limited to vitamin B12 (cyanocobalamin), biotin, and thiamine HCl, see EXAMPLE 13).

Variations of the DYV medium within which Alga X was initially cultured (e.g., alterations in macro- and micro-nutrients levels) have been successful in augmenting Alga X growth. For example, when the growth supplement, ALGA-GRO® Concentrated Medium (Carolina Biological Supply Company, Burlington, N.C.) was added to DYV medium, a significant increase in Alga X cell division rate per unit time was observed. ALGA-GRO® Concentrated Medium includes various micro- and macro-nutrients to support algal growth. In one embodiment of the present disclosure, a suitable amount of concentrated ALGA-GRO® supplement to DYV is in the range of about 1 to about 2 ml per liter of stock solution. However, it should be appreciated that other ranges are also within the scope of the present disclosure.

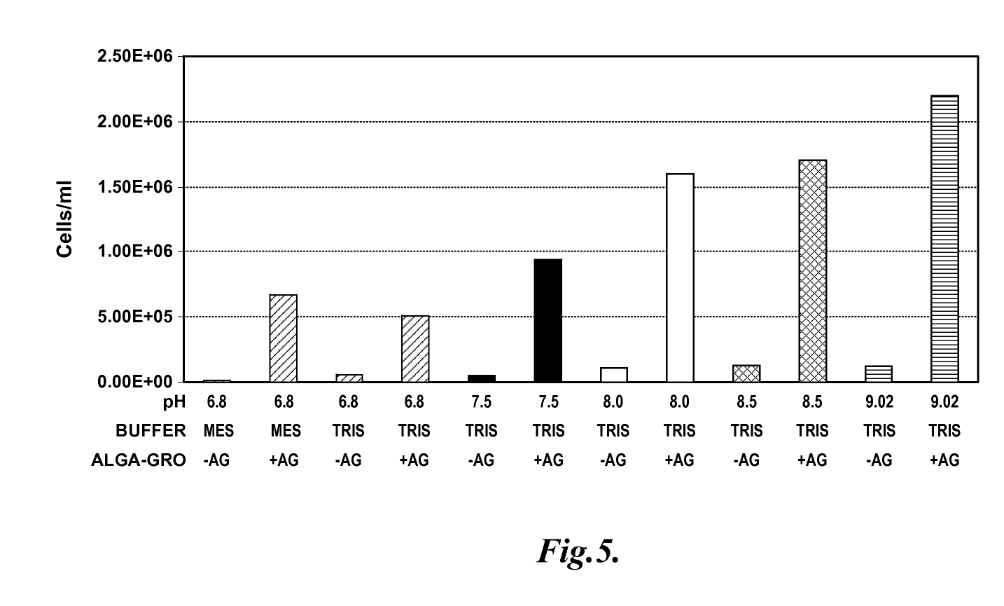

Additionally, growth of Alga X was discovered to be extremely responsive to pH changes. In that regard, referring to FIG. 5, there is a comparison of different culture densities resulting from the culture in media having varying pH, buffer solutions, and with/without algae growth supplement ALGA-GRO® (“+/−AG”). Significant growth augmentation can be seen as pH changes from 6.9 to 9.02. Moreover, the amount and type of buffer solution used also augments Alga X growth. In that regard, when 3.0 mM Tris buffer (which buffers pH in an alkaline range of about 7 to about 9) was substituted for the MES buffer (which buffers pH in an acidic range of about 4 to about 6) used in the original DYV medium, and the pH raised to over 8.0, good Alga X growth was achieved. Moreover, when buffered with AMPSO buffer (which buffers pH in an alkaline range of about 8.3 to about 10), excellent Alga X growth was achieved, as shown below in EXAMPLE 33.

In addition, pH has some effect on cell growth when the medium is buffered at an alkaline pH and when sodium bicarbonate is added as an additive, as shown below in Example 33. Without being bound by theory, the inventors believe that the cell culture typically receives carbon dioxide from ambient air. However, when bicarbonate ions are added to the medium (in the form of NaHCO₃), enhanced culture growth results, particularly in an alkaline-buffered medium. While sodium bicarbonate and alkaline pH buffering enhance cell growth in a distilled water based medium (CORE1, described in greater detail below), the use of waste water based medium (Clear, also described in greater detail below), in lieu of distilled water with added bicarbonate ions and pH buffering produces even more enhanced cell culture growth, as shown below in EXAMPLE 34.

However, pH has little effect on cell growth if other nutrients, for example, ALGA-GRO® concentrate, soil extract, or waste water, are not present in the growth medium. In one embodiment of the present disclosure, the medium includes pH in the range of about 6.8 to about 10. In another embodiment of the present disclosure, the medium includes pH in the range of about 8 to about 10.

In addition to nutrients and pH, experiments show that a small change in salinity, for example, effected by the addition of NaCl (about 3.0 mM) in the growth medium may alter the growth response and/or the fatty acid profile of Alga X. In particular, high salinity may inhibit the growth response of Alga X. In that regard, the inventors found that at 64 mM salt content, the Alga X cells did not survive. However, it was found that Alga X exhibits maximum growth at 8.0 mM level of NaCl (see EXAMPLE 12). Thus, this concentration was used in medium developed for the Alga X. Therefore, in one embodiment of the present disclosure, the medium includes salinity in the range of about 0 mM to about 32 mM. In another embodiment of the present disclosure, the medium includes salinity in the range of about 0 mM to about 16 mM. In yet another embodiment of the present disclosure, the medium includes salinity in the range of about 0 mM to about 8 mM.

Therefore, nutrition, pH, and salinity may be optimized to maximize algal growth during the selective culture of algae for biostock. In view of these findings, a new medium, RAC1, was developed that is composed of DYV ingredients, supplemented with 1.2 ml/liter of ALGA-GRO® concentrate (see EXAMPLE 13), 8 mM NaCl, and buffered with 3.0 mM Tris at pH 8.5. This medium supported the rapid growth of Alga X when cells were cultured at varying temperatures.

Soil extracts (“SEM”) have also been found to be suitable sources of micro- and macro-nutrients to support algal growth, either in addition to or as suitable replacements for ALGA-GRO® Concentrated Medium. While not wishing to be bound by theory, it is believed by the inventors that macro- and micro-nutrients in the soil extracts contribute to enhanced algal growth. Soil extracts are prepared from soil, compost or manure materials, as described in EXAMPLE 14 below. In the examples described herein, soil was collected from a greenhouse in Seattle, Wash. The advantage of using soil extract in lieu of ALGA-GRO® Concentrated Medium is not only in cost savings, but also in growth of Alga X.

An alternative medium, designated CORE1 (EXAMPLE 15), substituted ALGA-GRO® concentrate with soil extract. Different volumes of “soil extracts” (see EXAMPLE 14, soil extracts indicated as SEM) were added to 100 ml of CORE1. The data shows that such inexpensive “soil extract” supplements can substitute for ALGA-GRO® in supporting vigorous Alga X cell growth if used in proper amounts. In CORE2 medium (see EXAMPLE 16), different ingredient amounts were optimized over CORE1, for example, NH₄Cl, NaNO₃, beta-glycerophosphate, etc. Comparative algal culture growth curves show that RAC1 and CORE1 media achieve significantly improve culture growth over DYV.

In addition to soil extracts, waste water has also been found to be a suitable source of micro- and macro-nutrients to support algal growth, including but not limited to human waste water, cow waste water, horse waste water, and other waste water streams. Waste water generally contains additional nutrients, such as phosphates, ammonia, and/or trace elements (such as iron and zinc), which supplement the growth of Alga X. While not wishing to be bound by theory, it is believed that macro- and micro-nutrients in the waste streams contribute to enhanced algal growth.

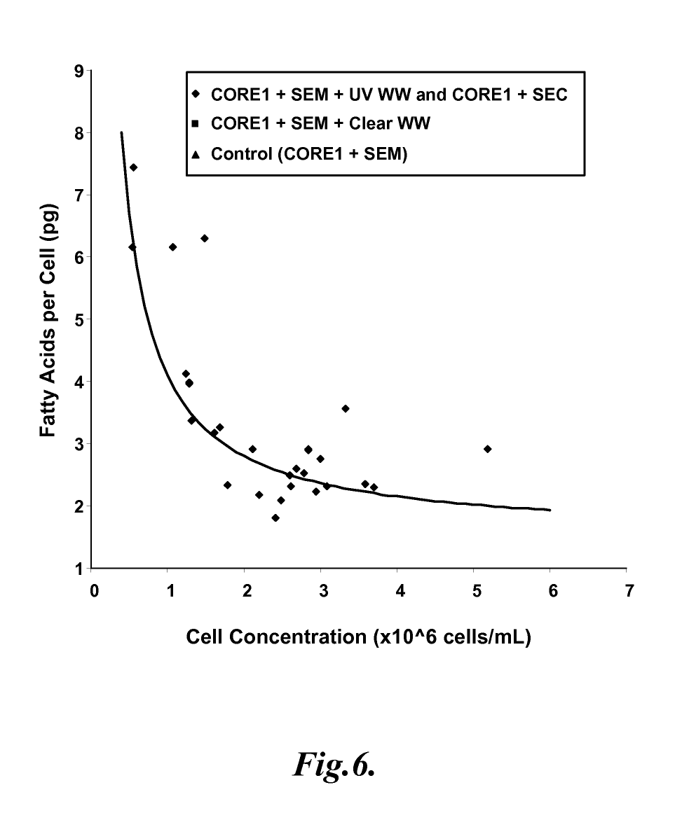

Notably, human waste water has been found to achieve very good results without the need for any additional medium ingredients. The added advantage of using human waste water is that no costs are incurred for water or other medium ingredients. In several non-limiting examples, human waste water was acquired from Sequim Water Treatment Plant in Sequim, Wash., USA. Various samples were obtained prior to UV treatment and after UV treatment at the waste water plant, as described in EXAMPLES 20 and 21 below. As seen in FIG. 6, human waste water prior to UV treatment (“Clear”) is a better growth medium additive for Alga X than UV treated waste water (“UV”), and both Clear and UV achieve increased fatty acid per cell over the control medium (CORE1+SEM). Comparative culture growth tables for various media, including DYV, RAC1, CORE1+horse manure extract, CORE1+organic soil extract, CORE1+SEM, CORE1+SEC (cow dairy waste extract), and CORE1+Clear (human waste water)+SEM, as seen in EXAMPLES 27 and 28 below.

Referring to FIG. 6, the plot shows that younger cells generally tend to have higher lipid or fatty acid contents (measured in fatty acids per cell in picograms) than older cells. Therefore, as the cell concentration (measured in cells per ml) of an algal culture increases, the amount of fatty acids per cell tends to decrease. Notably, the experimental medium including CORE1, SEM, and Clear human waste water have a significantly higher initial fatty acid content than (1) CORE1+SEM+UV waste water, (2) CORE1+SEC, or (3) control (CORE1+SEM), which means that with time and as the culture density increases, the fatty acid content in the individual cells still remains high for a medium including CORE1, SEM, and Clear human waste water.

It has been observed using mass spectroscopy that a shift in lipid amount per cell occurs as a culture ages. For example, spectroscopic analysis of extracted lipids show that cells in logarithmic growth phase (5.40×10⁵ cells/ml) contained 6.15+/−0.03×10⁻¹² gram lipid per cell, while those in stationary phase (5.18×10⁶ cells/ml) had 2.92+/−0.03×10⁻¹² g/cell. A signal decline, indicating a loss of lipid per cell for the populations was also obtained using flow cytometry. Data show a flow cytometer mean population signal for BODIPY 505/515 dye stained cells (described in detail below) of 343.6 fluorescent units (FU) for “log phase” (i.e., when the cells are growing at their most rapid rate of their growth) and 132.0 FU for stationary phase cultures (background was 2.31 FU).

A lipid analysis for Alga X cultures in various media including various components, such as trace metals, vitamins, salt, soil extract (SEM), pH buffer, cow dairy waste extract (SEC), human waste water (Clear WW or UV WW), acetate, phosphate, and nitrogen, is seen in EXAMPLES 9-28 below.

In accordance with embodiments of the present disclosure, a suitable medium includes water, an alkaline buffer solution, a trace metal ion solution, a vitamin solution, phosphate, and nitrogen. In accordance with embodiments of the present disclosure, the metal ions in the trace metal solution may include manganese, zinc, cobalt, molybdenum, vanadium, and selenium, 4d metals from the Periodic Table of Elements, calcium, potassium, magnesium, sodium, and lithium, and mixtures thereof.

In accordance with embodiments of the present disclosure, the vitamin solution may include vitamin B12, biotin, and thiamine HCl, and mixtures thereof. In accordance with embodiments of the present disclosure, the medium may include other nonmetal ions, such as silicon, selenium, bromine, and iodine, and mixtures thereof. In accordance with embodiments of the present disclosure, the medium may also include acetate, boron ions, and B-glycerolphosphate.

In accordance with embodiments of the present disclosure, the growth media may further include nutrition selected from the group consisting of algal growth freshwater medium formula, soil extract, waste water, and mixtures thereof. In accordance with embodiments of the present disclosure, the water in the growth medium may be selected from the group consisting of fresh water and waste water, such as human waste water, either UV of Clear.

For large-batch cultures (more than 1 liter), water-washed air was bubbled through or across the medium. No mechanical mixing was necessary. High-lipid producing algae may be cultured by the conventional culture such as batch culture, semi-batch culture or continuous culture in the usual suspension system. In the present disclosure, the aeration is conducted by air or mixed gas. Although the reaction vessel employed in the present disclosure may be any bubbling type with stirrer or without stirrer. Usually growing algae without mandatory mechanical stirring is preferable because costs are reduced.

In accordance with embodiments of the present disclosure, the cells may be grown in a semi-continuous batch culture for optimization. Semi-continuous batch culture or “bumping” is a procedure in which after a certain target is reached (e.g., after a certain number of days or when a certain target culture density has been reached), a portion (e.g., about half) of the culture is removed from the vessel and replaced with fresh medium. The purpose of culturing in this manner is to harvest cells when they are still within the log growth phase (i.e., when the cells are growing most rapidly), so that the lipid content per cell will continue to remain elevated. In that regard, over time lipid content per cell generally decreases as a culture ages; however, lipid content per cell generally remains high during the log phase of culture growth.

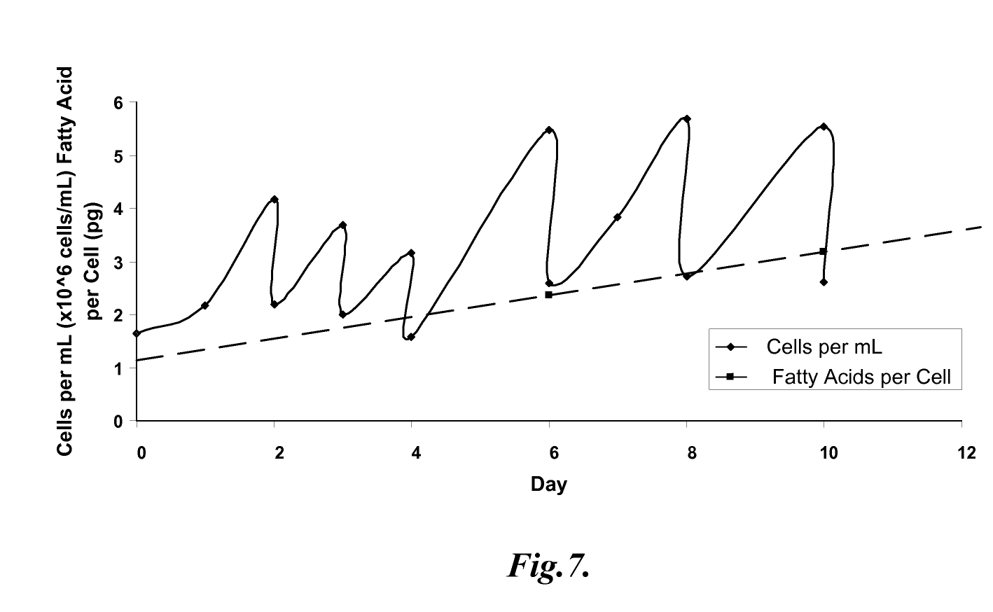

Referring to FIG. 7, a semi-continuous batch culture growth curve for Alga X is shown. Some interesting aspects of the experiment can be seen from the growth curve. First, optimal growth appears to be dependent on the type of medium in which the cells are grown. Second, cells in semi-continuous batch culture appear to have been selected for rapid growth, for they are able to maintain a high replication rate within a short time period, e.g., two days or less. Third, fatty acids per cell tend to increase with each successive bump, meaning that the cell culture is being trained or selected to produce more fatty acids per cell. Therefore, cells in the semi-continuous batch culture will produce more lipids in a shorter period of time than cells in a batch culture.

In accordance with embodiments of the present disclosure for growing an algal culture, the culture is bumped by removing a portion of the algal culture, for example, 50% of the algal culture, and replacing the culture with fresh medium. In one embodiment, the algal culture is bumped during the log phase of culture growth.

Lipid profile results for another semi-continuous batch culture or “bump” experiment are provided below in EXAMPLE 24. Comparison of lipid production for a culture that was harvested every other day for 8 days while being maintained as a semi-continuous batch culture versus a culture that was allowed to grow for 8 days straight, then harvested in its entirety, showed that the semi-continuous batch culture produces approximately a 3.5 to 4-fold more lipids during the culture period. Notably, the amount of lipids per cell increases as the semi-continuous culture is maintained. The value of lipids per cell in a semi-continuous culture is thus greater than that of a batch harvested culture.

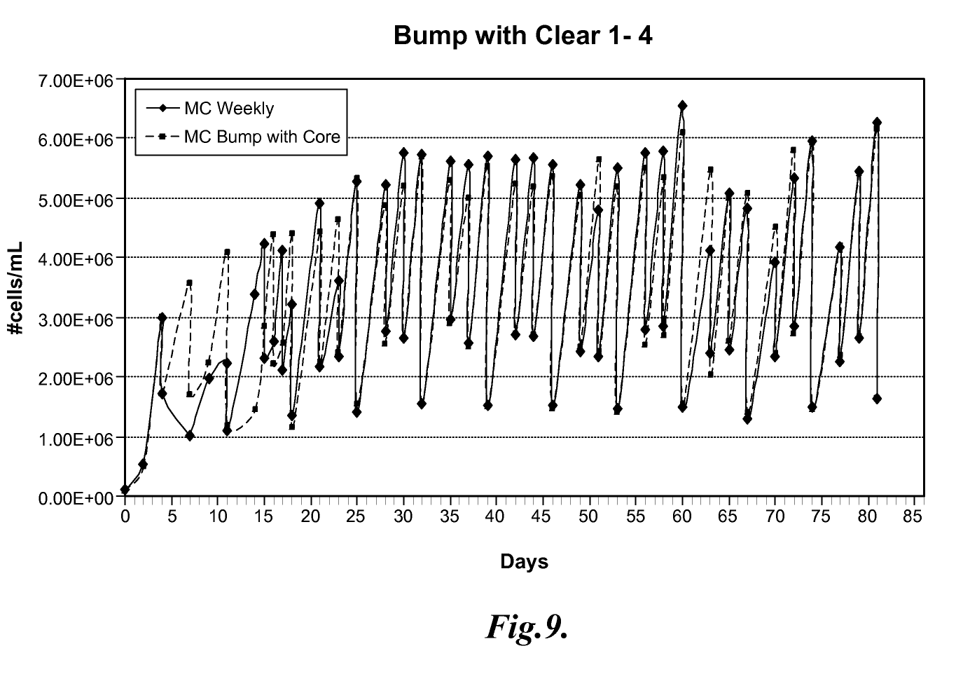

Referring to FIG. 9, another semi-continuous batch culture growth curve for Alga X is shown. Here, the inventors have shown that the culture may be bumped for extended periods of time of at least 80 days, and perhaps indefinitely, which is advantageous for commercial viability. The conditions for the semi-continuous batch culture growth are described below in EXAMPLE 35.

### Lipid Analysis

FAME ionization detection and GC/MS studies of cells from both small and large-scale cultures indicated that various conditions for Alga X growth impact the quality and composition of lipids obtained. While lipid profiles are dynamic, factors in the growth cycle that impact the quality and composition of lipids obtained include but are not limited to light intensity, light cycle, temperature, nutrients, salinity, pH, water source, and harvest rate.

Regarding temperature, data suggest that cells grown at either 18° C. or 22.5° C. maintain similar lipid profiles (see EXAMPLE 7). The percentage of unsaturated lipids present in cells grown at these temperatures can be very high, for example, greater than 80%. In contrast, cells grown at a higher temperatures (e.g., 24° C. or 29° C., see EXAMPLE 8) saturate their lipid chains, and also modify lipid chain length: C16:0 and C18:0 levels markedly increased at 24° C., while C18:5, C18:4, C20:4n6, C20:5, and C22:6 levels decreased dramatically when compared to cells grown at lower temperatures. Therefore, omega 3 and 6 fatty acids tend to increase with temperature deviations from the normal growth conditions, as do saturates, but the cells seem to acclimate to temperature changes by about day 6.

Changing pH increases growth rate as seen in FIG. 5, but also impacts the distribution of fatty acids generated by the cell. The data suggests that living and or dividing cells increase pH and dead or dying cells lower pH. The data in EXAMPLE 17 below suggests that an increase in pH not only increases cell growth rate, but also impacts the distribution of fatty acids in the algal cells.

Different media also impact fatty acid distributions. Clear waste water with CORE1 seems to produce good fatty acid profiles compared to the control medium (CORE1+SEM) (see EXAMPLE 20). In that regard, limiting core increases lipid per cell by upwards of 75%. Fatty acid profiles, however, remain largely unaffected by this increase in total lipids, although a slight increase in saturated fatty acids is observed. Clear waste water with no other media additives provides fatty acids per gram dry weight marginally higher than control (CORE1+SEM), but lipid profiles remain very similar. See EXAMPLE 19. On the other hand, the addition of UV-treated wastewater to CORE1 caused a slight increase in lipid per cell over control. Fatty acid profiles remain unaffected in any significant way. See EXAMPLE 21.

Fatty acids per cell vary only slightly with increasing dairy waste extract in the media compared to the control (CORE1+SEM), increasing with increasing dairy waste on day 3, but decreasing with increasing dairy waste on day 6. Profiles remain constant with changing dairy waste extract concentration, with little change in omega 3 and 6 and saturated fat composition. See EXAMPLE 18.

Regarding phosphate as a medium additive, fatty acid productivity increases slightly with increasing KH₂PO4 by day 6, though the increase is small and may or may not be significant. Lipid profiles remain fairly constant with changing phosphate levels. See EXAMPLE 25. Lipid data for varying B-glycerolphosphate levels is not available.

Regarding salt as a medium additive, low NaCl concentration appears to increase saturated fatty acids over the control without NaCl. See EXAMPLE 12. Salt addition to the medium can cause a stress response reflected in a change in the saturated to unsaturated fatty acid distribution. Notably, a low NaCl concentration (e.g., 8 mM) appears to increase saturates over no NaCl and higher concentrations. Omega 3 and 6 fatty acids appear to remain substantially consistent across different NaCl concentrations.

Regarding nitrogen as a medium additive, lipids per cell is little affected by increased nitrogen in the media; however, lipids per cell may be slightly suppressed by very high levels of nitrates. High nitrate levels increase omega 3 and 6 fatty acid content, though saturated fatty acid content appears to remain unaffected. C18s suppressed by high nitrate concentration. Cells appear to acclimate by about day 6. See EXAMPLE 26.

Regarding acetate as a medium additive, increased acetate caused a slight increase in fatty acids, particularly in the exponential phase, which leads to an overall increase in productivity. Omega 3 and 6 fatty acids are slightly suppressed with increased acetate, while saturated fats remain unchanged. Fatty acid profiles appear to be similar across all concentrations of acetate. See EXAMPLE 23.

Regarding the bump or semi-continuous batch culture experiment, fatty acids per cell appear to stabilize after bump, with lipid profiles of post-bump samples closely resembling those of day 6 controls. Control medium at day 9 in the bump experiment had a high fatty acid per cell content, likely because the cells had not yet divided. See EXAMPLE 24.

Regarding light, omega 3 and 6 fatty acids appear to increase with decreasing light levels, while saturated fatty acids appear to decrease with decreasing light levels.

As described above regarding light/dark cell cycles, lipids per cell begins low in the dark, and then increases throughout the day until L10, even while the cells are dividing. Omega 3 and 6 fatty acids decrease and saturated fats increase as lipid content increases, as is to be expected for growing liposomes. This fatty acid data agrees with confocal microscopy data taken concurrently. See EXAMPLE 5.

### Methods and Media for Selectively Adapting Algal Cultures

An algal population contains individual cells that range from low to high in their ability to synthesize lipids. Selecting those cells capable of high lipid generation and using those cells to initiate new algal populations with a shifted (higher) mean lipid production capacity was optimized through a high-throughput method of screening for algae with elevated lipid content.

Because some algae are unicellular, it is possible to sort individual cells from a population and select those with the highest lipid concentration for further culturing. Although molecular cloning techniques may be used to generate cells with high lipid content, the use of classic selection techniques avoids the debate experienced in the genetically modified food crop industry. Indeed, that a high-lipid algal culture may be selected via a high through-put selection method, as described herein, makes this technique an attractive and commercially viable approach to the generation of algae that can be used in biofuels production.

Because algal populations are large and cell division is rapid, Darwinian evolution can be “directed” in a laboratory setting, producing cells with unique, selected attributes. Physiological cues are used to drive the selection of new population genotypes rather than using a direct, molecular manipulation of targeted genes within the organism. Cell sorting may be achieved via fluorescent activated cell sorting (FACS) analysis. Similar to cells being size sorted by their chlorophyll autofluorescence signal, cells can be separated by lipid content by the strength of the fluorescent signal of the lipophilic dye that has been added to the cell. The small nature of the cells chosen and the localization of lipids in distinct vesicles allow for the quick sorting of algae containing different lipid quantities. Cells with the highest lipid content can be easily collected in test tubes.

The algae chosen for the present study, Alga X, is an excellent choice for developing lipid-screening technology via flow cytometry because it is small, round, and has highly localized lipid bodies within the cell. It must be noted, however, that the selection methodology may be applied to other unicellular algae having detectable lipid content. Other relatively small algae may be similarly screened and selected according to the methods of the present disclosure. For example, marine Isochrysis, Nannochlopsis, Pinguiococcus, and freshwater Chromulina and Chrysosaccus, may be suitable for high throughput, high lipid selection. Larger algae (e.g., some diatoms) may not be suitable because large cells may clog flow cytometric equipment.

The process used for generating high lipid-producing cultures is as follows. A sample from an algal mother culture is “sorted” using flow cytometry on an in Flux® cell sorter (Cytopeia Inc., Seattle, Wash.). In this step, cellular lipid content is monitored using the vital dye Nile Red (Sigma-Aldrich, Providence, R.I.) or another suitable dye.

Individual cells with the highest lipid profile (e.g., highest 0.5% lipid profile) are selected from the general population, and each individual cell is deposited into a separate well of a 96 well plate that contains 150 micro-liters of algal growth medium. Each cell in its individual mini-growth chamber serves as a progenitor of a new algal population with unique genetic identity. Plates containing the developing new populations are incubated under appropriate light and temperature conditions to augment algal growth.

Next, the plates are read in a Victor™ plate reader (PerkinElmer Inc., Waltham, Mass.) where cell number (optical density) and lipid content (using a lipophilic fluorescent lipid dye) are measured to select for strains that have both rapid growth and elevated lipid profiles. Populations of algae having these attributes are then cultured in larger volume, and the process repeated. If, in further experimentation, it is found that specific environmental conditions augment lipid biosynthesis, then these conditions are imposed as a further driver in this selection process. This process of sorting, and new culture initiation continues, generating new strains or cultures that are able to synthesize lipids in excess of the original parental or “mother” culture. The newly developed cultures are then tested for not only lipid quantity but for lipid quality as well.

A new dye for monitoring the storage of lipids within live algal cells includes using a fluorescent green dye, BODIPY 505/515 (4,4-difluoro-1,3,5,7-tetramethyl-4-bora-3a,4a-diaza-s-indacene, MW 248). Vitally-stained algal cells with large lipid bodies can then be quickly isolated using a fluorescent cell sorter. This rapid selection process can be used to assist in the directed evolution of algal strains for biofuels and other biotechnological applications.

BODIPY 505/515 is a neutral fluorophore that is highly lipophilic. This high quantum yield dye has been used previously to vitally stain lipid-containing yolk platelets in living zebrafish embryos. A similar dye, BODIPY 493/503, has been used to stain lipid droplets in mammalian cells.

In the past, algal cells stained with the lipophilic dye, Nile Red, have been analyzed and separated using a cell sorter, to rapidly isolate cells that possess the largest lipid bodies. The drawback of Nile Red is that it is a carcinogen. In addition, Nile Red is not photo-stable, in that it quickly fluorescently quenches or photo-bleaches when exposed to light, and it is impaired by the presence of chlorophyll. In contrast, BODIPY 505/515 is non-toxic and thereby suitable for human or animal consumption. Moreover, BODIPY 505/515 is photo-stable.

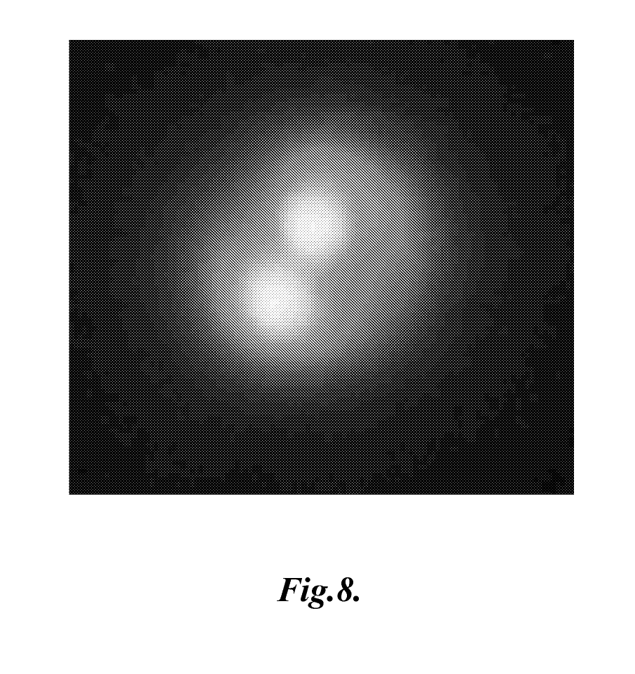

When added to a culture of algal cells, 1-10 micro-liters of BODIPY 505/515 stains intracellular lipid bodies within minutes. (Preparation of a BODIPY 505/515 stock solution is described below in EXAMPLE 30.) BODIPY 505/515 accumulates in lipidic compartments by a diffusion-trap mechanism. The dye has a high oil/water partition coefficient, which allows it to cross cell membranes and organelle membranes easily. When viewed under a fluorescent microscope, the background fluorescence of BODIPY 505/515 in the bath solution is slightly visible. Despite the slight background fluorescence, lipid bodies within live algal cells are intensely labeled and can be easily seen using fluorescein-type optics or confocal microscopy (see FIG. 8). BODIPY 505/515 has an excitation maximum of 505 nm, and an emission maximum of 515 nm. Chloroplasts exhibit moderate red fluorescence under the same blue excitation. This fluorescence arises from endogenous carotenoid and chlorophyll molecules located within the chloroplasts.

Notably, BODIPY 505/515 is able to vitally-stain lipid bodies in all alga species, even those that possess thick cell walls. In that regard, BODIPY 505/515 has been shown to vitally-stain lipid bodies in other algal genus/species/taxon in addition to Alga X (e.g., Chlamydomonas sp., Chlorophyceae; Emiliania huxleyi, Haptophyceae; Thalassiosira pseudonana, Bacilliariophyceae; Prorocentrum micans, Dinophyceae; and Mallomonas splendens, Synurophyceae). Single-cell and filamentous species of algae continue to grow well in the continuous presence of micromolar concentrations of the dye for days (data not shown).

Fluorescently labeled cells with large lipid bodies can be isolated using the fluorescent cell sorter and used to successfully seed new algal cultures. Fluorescently labeled cells have a signal/noise ratio of 50-200 above background fluorescence, depending on the BODIPY 505/515 dye concentration used. Thus, BODIPY 505/515 can be used to assess and monitor lipid stores within live algal cells without appreciable photodamage. BODIPY 505/515 can be used to characterize the diurnal rhythm of lipid body size within algal cells or decline in lipid quantity per cell as a culture moves from one life history phase to another. Such life cycle information may be of direct application to enhancing algae fatty acid yields in commercial algae farms.

Therefore, in accordance with embodiments of the present disclosure, a method of selectively generating an algal culture having an improved identification property includes obtaining a first algal culture having an identification property having a first value, isolating the first algal culture in a first growth medium, incubating the first algal culture in the first growth medium to provide a second algal culture, and sorting the second algal culture to select algal cells having the identification property having a second value to provide a sorted portion of the second algal culture. The identification property may be a high lipid content, a high biomass content, rapid growth rate, fatty acid profile, and combinations thereof. The first and second values may be ranges of values, for example, a lipid content in a range, or a lipid content greater than some number. The second value may be improved over the first value. The second algal culture may be sorted using flow cytometry and a lipophilic dye. It should be appreciated that the first and second growth media may be the same or different.

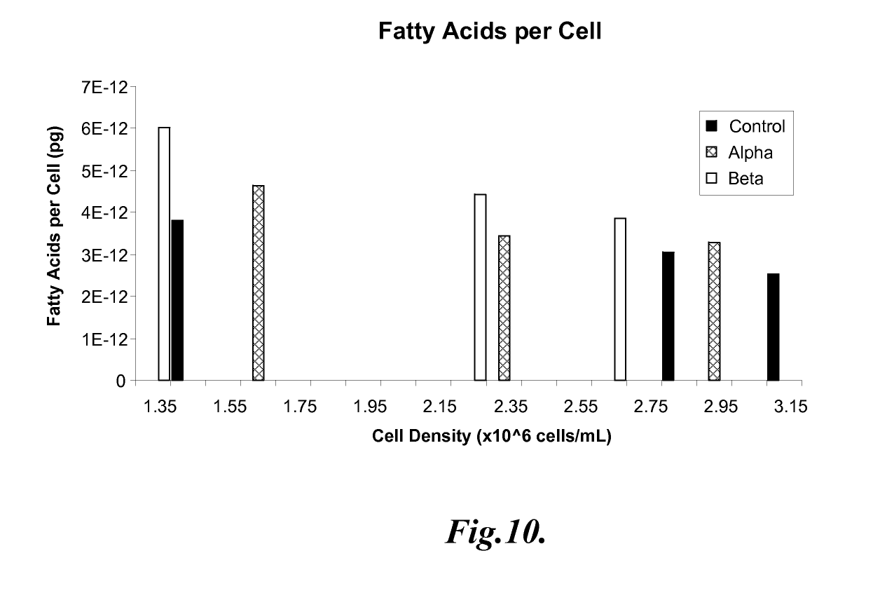

In another embodiment, the algal culture may be sorted multiple times. In one experiment, Alga X cells were sorted using a flow cytometer, selecting approximately 0.5% of the population having the highest lipid content. As described in EXAMPLES 31 and 35 and FIG. 10 below, an increase of 14% lipid content in the cells was observed after three sortings. In accordance with one embodiment of the present disclosure, at least 5% increase in lipid content can be achieved with cell sorting. In accordance with one embodiment of the present disclosure, at least 10% increase in lipid content can be achieved with cell sorting. In accordance with another embodiment of the present disclosure, at least 20% increase in lipid content can be achieved with cell sorting. In accordance with yet another embodiment of the present disclosure, at least 30% increase in lipid content can be achieved with cell sorting. In summary, there appears to be a significant increase in the amount of lipid per cell when cells are subject to sequential selection using flow cytometry.

Moreover, in accordance with one embodiment of the present disclosure, an increase of in saturate fatty acid content of about 15% to about 20% can be achieved with cell sorting. In accordance with another embodiment of the present disclosure, an increase of in saturate fatty acid content of about 20% to about 25% can be achieved with cell sorting. In accordance with yet another embodiment of the present disclosure, an increase of in saturate fatty acid content of greater than about 25% can be achieved with cell sorting.

### Products Derived from the Algal Culture

As mentioned above, algae fatty acids have a variety of commercial and industrial uses, and are extracted through a wide variety of methods. When an alga is dried, it retains its oil content, which then can be “pressed” out with an oil press. Many commercial manufacturers of vegetable oil use a combination of mechanical pressing and chemical solvents in extracting oil. Algal oil can also be extracted using enzymatic extraction, osmotic shock, supercritical fluids, or ultrasonic-assisted extraction. The waste products from chemical crushing may then be used as fuel (analogous to wood), as an additive to animal feed, or as compost. In the present context, the lack of a cell wall in Alga X augments lipid retrieval from this organism and reduces the amount of extraneous byproduct.

Algal cells that synthesize large amounts of lipids are needed as biostock in the production of biodiesel. The selection and rapid identification of algae that can serve as the progenitors of new oleaginous strains is of great market importance to the biodiesel industry. In accordance with embodiments of the present disclosure, a lipid mixture product obtained from Alga X cells includes the following: (a) C14 in an amount in the range of about 14 to about 25 weight percent of the total lipid content; (b) C16 in an amount in the range of about 17 to about 26 weight percent of the total lipid content; (c) C18 in an amount in the range of about 29 to about 57 weight percent of the total lipid content; and (d) C20 and greater in an amount in the range of about 9 to about 30 weight percent of the total lipid content.

### EXAMPLES

The following examples are directed to different experiments regarding Alga X. In some examples below cells in the experimental flasks are obtained from a mother culture (MC) of Alga X, which is generally maintained at standard conditions (100 uE/m2/sec, 20° C., in a medium of CORE1+SEM). The mother culture is generally about one week old and has a culture density of about 2×10̂6.

### Example 1

### Lipid Content

Different types of algal cells were tested for lipid content. Comparative data for lipid content in algal cells is provided below, showing that Alga X has a higher lipid content than other algal cells. (Comparative data adapted from Manual on the Production and Use of Live Food for Aquaculture, Section 2.4: Nutritional Value of Micro-Algae, available at http://www.fao.org/docrep/003/w3732e/w3732e07.htm.) Lipid content below was measured by using the Bligh-Dyer solvent extraction process for lipids. (As mentioned above, when measured by mass spectroscopy, Alga X has a fatty acid content of 2-20×10̂12 g fatty acids per gram dry weight.)

| Cell Type | Lipid/Dry Weight (mg/g) |
|---|---|
| Alga X | 350 |
| Chaetoceros | 160 |
| Chroomonas | 120 |
| Cyclotella | 183 |
| Dunaliella | 150 |
| Isochrysis | 230 |
| Nannochloropsis | 180 |
| Nannochloris | 210 |
| Phaeodactylum | 112 |
| Pavlovia | 120 |
| Tetraselmis | 170 |

### Example 2

### Lipid Type

Different types of algal cells were tested for lipid type in the lipid content, i.e., saturated fatty acids, for example, for biodiesel applications and unsaturated fatty acids, for example, for pharmaceutical and nutraceutical applications. Comparative data for lipid type in algal cells is provided below. Lipid type was measured by mass spectroscopy.

| | Percent of Total | | |
|---|---|---|---|
| | Lipids By Weight | | |
| | Biodiesel Fatty | Nutraceutical | |
| Cell Type | Acids | Fatty Acids | Other |
| Alga X | 69 | 31 | <1 |
| Phaeodactylum | 60 | 33 | 7 |
| Nannochloropsis | 56 | 44 | <1 |
| Dunaliella | 53 | 39 | 8 |
| Chlorella | 46 | 53 | <2 |
| Porphyridium | 36 | 64 | <1 |

### Example 3

### Light Intensity

Various light intensities were tested for Alga X samples grown in CORE1+SEM medium at 20C with 12L/12D light cycle. Experimental variable was light intensity using 20, 40, 60, and 100 ue/m2/sec full spectrum light bulbs. Culture density in cells per milliliter was compared as an indicator of growth. As seen in tables below, all specimens of Alga X showed algal growth, with 100 ue/m2/sec showing the best growth. In addition, growth was tested at extreme light intensities of 3 and 160 ue/m2/sec. Cell growth was normal at 160 ue/m2/sec. Although cell growth was not significant at 3 ue/m2/sec, the cells did show an ability to adapt and survive extreme light fluctuations.

In summary, Alga X requires a cycle of lightness and darkness for optimal growth, with continuous light being suboptimal for growth, and the cells can grow well in an “artificial sunlight” program. Data also suggests that culture age (often affected for other algae by light attenuation) does not significantly affect fatty acid profile. In general, omega 3 and 6 fatty acids increase with decreasing light levels, while saturated fats decreases with decreasing light levels. There is a significant advantage in having fatty acid profiles that remain relatively constant when cultures are exposed to different light quantities.

| | Light Intensity (ue/m2/sec) | | | |
|---|---|---|---|---|
| Day | 20 | 40 | 60 | 100 |
| day 0 | 2.00E+04 | 2.00E+04 | 2.00E+04 | 2.00E+04 |
| MC = 2.16E+06 | | | | |
| day 2 | 7.15E+04 | 2.46E+04 | 9.05E+04 | 9.08E+04 |
| day 3 | 9.47E+04 | 3.94E+04 | 1.79E+05 | 3.11E+05 |
| day 4 | 2.10E+05 | 9.07E+04 | 2.59E+05 | 6.69E+05 |
| day 6 | 7.47E+05 | 5.33E+05 | 1.97E+06 | 2.41E+06 |
| day 8 | 1.95E+06 | 1.93E+06 | 2.74E+06 | 3.07E+06 |
| day 10 | 2.84E+06 | 3.29E+06 | 3.68E+06 | 4.21E+06 |

| | Day | 20 uE | 40uE | 60 uE | 100 uE |
|---|---|---|---|---|---|
| cells/mL | | 2.00E+04 | 2.00E+04 | 2.00E+04 | 2.00E+04 |
| MC = 2.15E+06 | 3 | 9.47E+04 | 3.93E+04 | 1.79E+05 | 3.11E+05 |
| | 4 | 2.10E+05 | 9.07E+04 | 2.59E+05 | 6.96E+05 |
| | 6 | 7.47E+05 | 5.33E+05 | 1.97E+06 | 2.41E+06 |
| | 8 | 1.95E+06 | 1.93E+06 | 2.74E+06 | 3.07E+06 |
| Total w3 + w6 | 0 | 44.0 | 44.0 | 44.0 | 44.0 |
| (%) | 3 | X | X | X | 49.2 |
| | 4 | 49.3 | X | 52.7 | 50.5 |
| | 6 | 55.0 | 47.3 | 52.3 | X |
| | 8 | 56.3 | 54.3 | 52.5 | 49.1 |
| Saturates | 0 | 33.1 | 33.1 | 33.1 | 33.1 |
| (%) | 3 | X | X | X | 37.5 |
| | 4 | 34.6 | X | 36.2 | 40.0 |
| | 6 | 38.1 | 43.9 | 38.3 | X |
| | 8 | 34.2 | 36.8 | 35.3 | 37.1 |
| Lipid Profile | | | | | |
| C14s | 0 | 12.7 | 12.7 | 12.7 | 12.7 |
| C16s | | 26.3 | 26.3 | 26.3 | 26.3 |
| C18s | | 45.2 | 45.2 | 45.2 | 45.2 |
| C20+s | | 15.8 | 15.8 | 15.8 | 15.8 |
| C14s | 3 | X | X | X | 15.7 |
| C16s | | X | X | X | 22.3 |
| C18s | | X | X | X | 48.3 |
| C20+s | | X | X | X | 13.7 |
| C14s | 4 | 14.8 | X | 17.1 | 16.8 |
| C16s | | 23.2 | X | 19.1 | 21.4 |
| C18s | | 47.2 | X | 52.8 | 48.0 |
| C20+s | | 14.8 | X | 11.0 | 13.8 |
| C14s | 6 | 16.3 | 18.4 | 15.4 | X |
| C16s | | 20.9 | 24.5 | 21.4 | X |
| C18s | | 45.0 | 46.0 | 46.5 | X |
| C20+s | | 17.8 | 11.1 | 16.7 | X |
| C14s | 8 | 15.4 | 16.0 | 15.5 | 15.4 |
| C16s | | 19.0 | 20.1 | 20.3 | 21.7 |
| C18s | | 44.3 | 46.0 | 46.5 | 46.4 |
| C20+s | | 21.3 | 17.9 | 17.7 | 16.5 |

### Example 4

### Light Exposure

Six different photoperiods were analyzed using Alga X in a standard medium (CORE1+SEM). All cultures were grown at 100 ue/m2/sec and 20° C. Results for the following photoperiods are detailed below: (a) 12 hours of light and 12 hours of dark (12 L12 D); (b) 16 hours of light and 8 hours of dark (16 L8 D); (c) 8 hours of light and 16 hours of dark (8 L16 D); (d) 6 hours of light, 6 hours of dark, followed by 6 hours of light and 6 hours of dark (6 L6 D); and (f) continuous light (not included below). Of the six different photoperiods analyzed, all specimens of Alga X showed acceptable algal growth except for the continuous light photoperiod, with 12 hours of light and 12 hours of dark showing the best algal growth.

| Light/Dark Cycle | Start Cells/mL | End Cells/mL (Day 6) |
|---|---|---|
| 12L12D CORE1 + SEM | 1.00E+05 | 3.55E+06 |
| 16L8D CORE1 + SEM | 1.00E+05 | 2.78E+06 |
| 8L16D CORE1 + SEM | 1.00E+05 | 2.22E+06 |
| 6L6D CORE1 + SEM | 1.00E+05 | 2.56E+06 |

### Example 5

### Fatty Acid Cycle

A culture of Alga X was grown at a light intensity of about 100+/−10 ue/m2/sec at a 12 L/12 D light cycle at about 21° C. The alga was a first generation alga grown in medium including Clear, SEM, and CORE1. Appreciable increase in fatty acids per cell was observed between D11.5 (11.5 hours into the dark cycle) and L11.5 (11.5 hours into the light cycle), as seen in table below as well as in FIG. 4. Therefore, fatty acids per cell begins low in the dark (D11.5), and then increases throughout the day until L10, even while the cells are dividing (see EXAMPLE 7 below).

In addition, a general decrease in omega-3 and omega-6 fatty acids is seen between D11.5 and L11.5, and a general increase in saturated fatty acids is seen between D11.5 and L11.5, as is to be expected for growing liposomes. Quantities of C14, C16, C18, and C20+ also vary depending on the light/dark cycle. This fatty acid data agrees with confocal microscopy data taken concurrently.

| | | Day1 | Day 2 | Day 2 | Day 2 | Day 2 | Day 2 | Day 3 |
|---|---|---|---|---|---|---|---|---|
| | MC | D11.5 | L1 | L4 | L7 | L10 | L11.5 | L6.5 |
| fatty acids | 3.5E−12 | 4.12E−12 | 5.82E−12 | 7.07E−12 | 7.44E−12 | 9.45E−12 | 9.69E−12 | 6.29E−12 |
| per cell | | | | | | | | |
| ω3 + ω6 | 35.4 | 47.8 | 45.6 | 39.1 | 40.2 | 40.8 | 37.0 | 38.7 |
| saturates | 19.8 | 23.0 | 22.0 | 23.1 | 28.7 | 28.8 | 28.4 | 25.7 |
| C14 | 9.2 | 6.6 | 7.2 | 8.4 | 10.4 | 10.4 | 10.4 | 10.3 |
| C16 | 12.4 | 17.9 | 14.9 | 13.7 | 16.8 | 17.1 | 17.1 | 14.7 |
| C18 | 63.0 | 43.1 | 46.2 | 51.3 | 48.0 | 54.7 | 59.2 | 61.1 |
| C20+ | 15.6 | 32.4 | 31.7 | 26.6 | 24.9 | 17.9 | 13.3 | 14.0 |

### Example 6

### Cell Division Cycle

A culture of Alga X was grown in the conditions described above in EXAMPLE 5. An increase in cells/ml was observed with time. However, an appreciable increase in cells/ml was observed between L1 (one hour into the light cycle) and L4 (four hours into the light cycle) and continued until L10, as seen below as well as in FIG. 4.

| | | Day 1 | Day 2 | Day 2 | Day 2 | Day 2 | Day 2 | Day 3 |
|---|---|---|---|---|---|---|---|---|
| | MC | D11.5 | L1 | L4 | L7 | L10 | L11.5 | L6.5 |
| cells/ml | 1.00E+5 | 3.64E+05 | 3.46E+05 | 3.90E+05 | 5.58E+05 | 7.51E+05 | 7.17E+05 | 1.49E+06 |

### Example 7

### Temperature

Alga X cells were grown in a medium of CORE1 and SEM, under light intensity of 100 ue/m2/sec+/−10 for 16 L/8 D at various temperatures. The Alga X cells were collected for total lipid analysis at 0, 2, 3, 5, 6, 8, and 9 days for each temperature, 12° C., 18° C., and 22.5° C., as seen in the data below. The changes in percentages at different days are not highly significant, indicating that temperature change will not produce a large change in total lipid content. Omega 3 and 6 fatty acids tend to increase with temperature deviations from their normal growth conditions, as do saturates, but the cells tend to acclimate to temperature changes by day 6 in the growth cycle.

Movement of the cells, for example, from a 22.5° C. medium to a 12° C. medium can cause a stress response, which can be reflected in a small change in the saturated to unsaturated fatty acid distribution, whereas a large change in amount of omega-3 and -6 fatty acids is observed.

| | Temperature (° C.) | | |
|---|---|---|---|
| Day | 12 | 18 | 22.5 |
| day 0 | 1.00E+05 | 1.00E+05 | 1.00E+05 |
| MC = 2.35E+06 | | | |
| day 2 | 2.26E+05 | 4.75E+05 | 7.42E+05 |
| day 3 | 3.83E+05 | 1.00E+06 | 1.37E+06 |
| day 5 | 1.24E+06 | 1.98E+06 | 2.29E+06 |
| day 6 | 2.03E+06 | 2.57E+06 | 2.78E+06 |
| day 8 | 3.19E+06 | 3.68E+06 | 3.68E+06 |
| day 9 | 3.81E+06 | 4.08E+06 | 4.28E+06 |

| | Day | 12° C. | 18.5° C. | 22.5° C. |
|---|---|---|---|---|
| cells/mL | 0 | 1.00E+05 | 1.00E+05 | 1.00E+05 |
| MC = | 3 | 3.83E+05 | 1.00E+06 | 1.37E+06 |
| 2.35E+06 | 6 | 2.03E+06 | 2.57E+06 | 3.81E+06 |
| | 9 | 3.81E+06 | 4.08E+06 | 4.28E+06 |
| fatty acids/g | 0 | 0.209 | 0.218 | 0.209 |
| (mg) | | | | |
| Total w3 + w6 | 0 | 42.8 | 48.2 | 48.2 |
| (%) | 3 | 37.3 | 51.1 | 53.5 |
| | 6 | 52.7 | 43.6 | 50.1 |
| | 9 | 46.2 | 43.9 | 34.7 |
| Saturates | 0 | 33.1 | 33.1 | 33.1 |
| (%) | 3 | 37.4 | 33.1 | 37.2 |
| | 6 | 33.9 | 30.0 | 34.5 |
| | 9 | 22.1 | 21.3 | 29.5 |
| Lipid Profile | | | | |
| C14s | 0 | 13.2 | 14.3 | 14.3 |
| C16s | | 24.2 | 15.8 | 15.8 |
| C18s | | 45.4 | 50.1 | 50.1 |
| C20+s | | 17.4 | 19.8 | 19.8 |
| C14s | 3 | 11.8 | 12.7 | 14.5 |
| C16s | | 24.5 | 20.5 | 24.0 |
| C18s | | 41.7 | 42.1 | 42.1 |
| C20+s | | 21.2 | 27.9 | 19.5 |
| C14s | 6 | 16.5 | 14.5 | 13.5 |
| C16s | | 18.4 | 16.2 | 22.8 |
| C18s | | 40.9 | 41.4 | 44.5 |
| C20+s | | 24.2 | 27.9 | 19.2 |
| C14s | 9 | 18.4 | 16.2 | 14.2 |
| C16s | | 14.6 | 17.7 | 18.5 |
| C18s | | 46.8 | 48.0 | 53.8 |
| C20+s | | 20.2 | 18.1 | 13.5 |

### Example 8

### Extreme Temperatures

Alga X cells were grown in a medium of CORE1 and SEM, under light intensity of 100 ue/m2/sec+/−10 for 12 L/12 D at various temperatures. The Alga X cells were collected for total lipid analysis at 0 and 16 days for 4° C., 0 and 12 days for 24° C., and 0, 6, and 11 days for 29° C. The results below show that although not optimized for growth at these extreme temperatures, the cells can survive at extreme temperatures.

Temperature (° C.)

| Day | 4a | 4b | 24 | 29 |
|---|---|---|---|---|
| day 0 | 1.00E+05 | 1.00E+05 | 4.47E+05 | 9.37E+05 |
| day 6 | x | x | x | 7.26E+05 |
| day 11 | x | x | x | 8.92E+05 |
| day 12 | x | x | 2.96E+06 | x |
| day 16 | 9.51E+05 | 7.24E+05 | x | x |

### Example 9

### DYV Medium Recipe DYV medium was developed by Keller and Anderson, Provasoli-Guillard National Center for Culture of Marine Phytoplankton. To 950 ml distilled water (dH₂O), add the quantity of the stock solution as indicated below.

Autoclaved DYV medium produces extensive silica precipitation; therefore, silicate can be deleted from the recipe when it is not required by the alga. Make final volume up to 1 L with dH₂O. Adjust pH to pH 6.8 with NaOH, then autoclave. This medium may be prepared as a DYV(5×) stock by using five-times the nutrients (e.g., 5 ml of NaNO₃ stock solution).

| Quantity | | Compound | Stock Solution | |
|---|---|---|---|---|
| 1 | ml | MgSO₄•7H₂O | 50 | g/L dH₂O |
| 1 | ml | KCl | 3 | g/L dH₂O |
| 1 | ml | NH₄Cl | 2.68 | g/L dH₂O |
| 1 | ml | NaNO₃ | 20 | g/L dH₂O |
| 1 | ml | beta-glycerophosphate | 2.16 | g/L dH₂O |
| 1 | ml | H₃BO₃ | 0.8 | g/L dH₂O |
| 1 | ml | Na₂EDTA•2H₂O | 8 | g/L dH₂O |
| 1 | ml | Na₂SiO₃•9H₂O* | 14 | g/L dH₂O* |
| 1 | ml | FeCl₃•6H₂O | 1 | g/L dH₂O |
| 1 | ml | CaCl₂ | 75 | g/L dH₂O |

| | | | |
|---|---|---|---|
| 200 | mg | MES | — |
| 1 | ml | DY trace metal ion solution | see below |
| 0.5 | ml | f/2 vitamin solution | see below |

### Example 10

### DYV Trace Metal Ion Solution

The DYV trace metal ion solution was prepared as follows:

Weigh and dissolve the chemicals listed below individually: Measure 100 ml ddH2O into a 250 ml beaker, add a stir bar and begin stirring on a stir plate. Weigh the first chemical and add it to the water in the beaker. Stir until the chemical is completely dissolved. Label the contents of the beaker and set aside. Repeat steps for each of the six chemicals. Pour contents of each of the six 250 ml beakers into the 1000 ml beaker. Bring volume up to 1000 ml by adding ddH2O. Add stir bar and stir on stir plate until combined. Pour into a 1000 ml glass bottle, cover with screw top, and label contents. Store in 4° C. cold chamber or refrigerator.

| Quantity | | Compound |
|---|---|---|
| 200 | mg | MnCl₂•4H₂O |
| 40 | mg | ZnSO₄•7H₂O |
| 8 | mg | CoCl₂•6H₂O |
| 20 | mg | Na₂MoO₄•6H₂O |
| 2 | mg | Na₃VO₄•nH₂O |
| 2 | mg | H₂SeO₃ |

### Example 11

### F/2 Vitamin Solution for DYV

F/2 vitamin solution for DYV was prepared as follows: Add the components as indicated below to make final volume up to 1 L with dH₂O, autoclave and store in refrigerator. Alternatively, make final volume up to 1 L with dH₂O, then filter sterilize into glass containers and store in refrigerator.

Note: Vitamin B12 and biotin are obtained in a crystalline form. When preparing the vitamin B12 stock solution, allow for about 11% water of crystallization (for each 1.0 mg of Vitamin B12, add 0.89 ml dH₂O). When preparing the biotin stock solution, allow for about 4% water of crystallization (for each 1.0 mg of biotin, add 9.6 ml dH₂O).

Guillard & Ryther, 8 Canadian J. Micro. 229039 (1962); Guillard, in CULTURE OF MARINE INVERTEBRATE ANIMALS, 26-60 (Smith & Chanley, eds., Plenum Press, NY, 1975).

| | | Stock | Final Molar |
|---|---|---|---|
| Quantity | Compound | Solution | Conc. |
| 1 ml | Vitamin B12 | 1.0 g/L dH2O | 1 × 10-10M |
| | (cyanocobalamin) | | |
| 10 ml | Biotin | 0.1 g/L dH2O | 1 × 10-9M |
| 200 mg | Thiamine HCl | | 1 × 10-7M |

### Example 12

### Salt

Two experiments were run using 0, 8, 16, 32, and 64 mM NaCl salt concentrations in the Alga X culture medium. Alga cells were collected for lipid analysis at 0, 3, 6, and 9 days for each salt concentration. Because an Alga X culture can grow in a low salt content medium, the use of low-salinity brackish water is an acceptable culture medium. The resultant data provided below shows that an Alga X culture can grow in a low salt content medium, but that cell growth is depressed at 32 mM salt content. At 64 mM salt content, the Alga X cells did not survive.

Salt addition to the medium can cause a stress response reflected in a change in the saturated to unsaturated fatty acid distribution. Notably, a low NaCl concentration (e.g., 8 mM) appears to increase saturates over no NaCl and higher concentrations. Omega 3 and 6 fatty acids appear to remain substantially consistent across different NaCl concentrations.

| | | Salt Concentration (mM) | | | |
|---|---|---|---|---|---|
| Fatty Acids | Day | 0 | 8 | 16 | 32 |
| cells/ml | 0 | 1xE+05 | 1xE+05 | 1xE+05 | 1.E+05 |
| MC = | 3 | 1.12E+06 | 1.07E+06 | 1.11E+06 | 5.88E+05 |
| 2.52E+06 | 6 | 2.93E+06 | 2.90E+06 | 2.89E+06 | 2.59E+06 |
| | 9 | 3.74E+06 | 3.98E+06 | 4.32E+06 | 3.72E+06 |
| fatty | 0 | x | x | x | x |
| acids/cell | 3 | 5.20E−12 | 6.15E−12 | 4.81E−12 | 8.84E−12 |
| | 6 | 3.40E−12 | 6.78E−12 | 1.99E−12 | 3.22E−12 |
| | 9 | x | x | x | x |
| ω3 + ω6 | 0 | 48.7 | x | x | x |
| (wt %) | 3 | 33.3 | 32.3 | 35.5 | 32.9 |
| | 6 | 32.1 | 28.1 | 42.5 | 35.7 |
| | 9 | 36.4 | 34.9 | 35.9 | 34.4 |
| saturates | 0 | 36.5 | x | x | x |
| (wt %) | 3 | 53.5 | 48.7 | 46.6 | 47.3 |
| | 6 | 37.3 | 38.9 | 38.0 | 42.1 |
| | 9 | 27.3 | 30.2 | 26.4 | 26.8 |
| C14 | 0 | 14.2 | 14.2 | 14.2 | 14.2 |
| C16 | | 22.5 | 22.5 | 22.5 | 22.5 |
| C18 | | 43.9 | 43.9 | 43.9 | 43.9 |
| C20+ | | 19.4 | 19.4 | 19.4 | 19.4 |
| C14 | 3 | 20.3 | 17.8 | 24.2 | 18.7 |
| C16 | | 27.7 | 25.3 | 18.9 | 26.2 |
| C18 | | 37.0 | 42.8 | 42.7 | 39.6 |
| C20+ | | 15.0 | 14.1 | 14.2 | 15.5 |
| C14 | 6 | 14.3 | 8.6 | 13.4 | 17.6 |
| C16 | | 28.4 | 20.8 | 24.4 | 27.1 |
| C18 | | 44.6 | 55.7 | 42.2 | 35.5 |
| C20+ | | 12.7 | 14.9 | 20.0 | 19.8 |
| C14 | 9 | 12.9 | 14.7 | 12.4 | 13.5 |
| C16 | | 16.9 | 17.9 | 16.0 | 15.1 |
| C18 | | 59.0 | 55.3 | 59.4 | 60.0 |
| C20+ | | 11.2 | 12.1 | 12.2 | 11.4 |

### Example 13

### RAC1 Medium

The following components below were mixed and the final volume adjusted to 1 L with double-distilled water (ddH₂O). The pH was then adjusted to pH 8.5, and the medium autoclaved for 30 minutes on liquid cycle.

| Quantity | Compound | Stock Solution (g/L) | |
|---|---|---|---|
| 1 ml | MgSO₄ 7H₂O | 50 | g/L ddH₂O |
| 1 ml | KCl | 3 | g/L ddH₂O |
| 1 ml | NH₄Cl | 2.68 | g/L ddH₂O |
| 1 ml | NaNO₃ | 20 | g/L ddH₂O |
| 1 ml | beta-glycerophosphate | 2.16 | g/L ddH₂O |
| 1 ml | H₃BO₃ | 0.8 | g/L ddH₂O |
| 1 ml | Na₂EDTA 2H₂O | 8 | g/L ddH₂O |
| 1 ml | FeCl₃ 6H₂O | 1 | g/L ddH₂O |
| 1 ml | CaCl₂ | 75 | g/L ddH₂O |
| 2 ml | NaCl 4M solution | 225.6 | g/L ddH₂O |
| 1 ml | TRIS 3M solution | 363.42 | g/L ddH₂O |

| | | |
|---|---|---|
| 1 ml | DY trace metal ion solution | see above |
| 0.5 ml | f/2 vitamin solution | see above |
| 1.2 ml | ALGA-GRO ® concentrate | |

### Example 14

### Soil Extract

Soil was collected from a greenhouse in Seattle, Wash. A soil extract was used to supplement RAC1, as an alternative to ALGA-GRO® concentrate, and was prepared as follows:

(1) Use a weigh boat and an electric top-loading balance to weight out 250 g of soil.

(2) Carefully pour the soil into a 2.8 L Erlenmeyer flask. Discard weigh boat.

(3) Weigh 2.54 g of NaOH pellets using a different weigh boat.

(4) Transfer the NaOH pellets into a 40 ml glass beaker.

(5) Add approximately 5-10 ml of ddH2O into the 40 ml beaker with the NaOH pellets (just enough ddH2O to dissolve all of the NaOH).

(6) Gently stir the NaOH pellets until all are dissolved into solution.

(7) Measure out 1 L of ddH2O using a 1 L graduated cylinder.

(8) Pour the 1 L of ddH2O into the 2.8 L flask with the soil.

(9) Pour the NaOH solution into the 2.8 L Erlenmeyer flask with soil and 1 L ddH2O.

(10) Swirl the 2.8 L flask to mix the soil, water, and NaOH solution.

(11) Cover the 2.8 L flask with aluminum foil and run a piece of autoclave tape across the flask's opening and over the aluminum foil to secure the foil onto the flask.

(12) Put the 2.8 L flask and its contents into an autoclave oven. Run the autoclave in ISOTHERMAL cycle (0 atm pressure, 103 degrees C.) for 90 minutes.

(13) After 90 minutes, remove the autoclaved flask from the autoclave and leave it out to cool down to room temperature (about 12-16 hours).

The soil mixture was filtrated and the soil extract was diluted as follows:

(1) Before beginning filtering the soil mixture, rinse the filter bag three times with tap water and three times with ddH2O.

(2) Pour the entire soil mixture into the filter bag and wait for the soil extract to filter through the filter bag and into a 1 L autoclavable bottle via a funnel.

(3) Squeeze the filter bag with your hands to squeeze out the soil extract from the soil debris. Discard the soil debris when done.

(4) Label this bag-filtered extract and note that this is the concentrated stock.

(5) Dilute the concentrated soil extract stock 1:50 (for every 1 ml of concentrated soil extract, there are 49 ml of ddH2O to make a total volume of 50 ml).

(6) Take the diluted soil extract and filter again with a coffee filter using a Buchner filter funnel, a 1 L filtering flask, and an aspirator (vacuum filtration).

(7) Autoclave diluted working stock on ISOTHERMAL cycle for 90 minutes.

(8) Discard any unused concentrated stock.

The media was stored as follows:

(1) After autoclaving, always let media cool down to room temperature.

(2) Store soil extract in the 4 degree C. cold chamber or refrigerator.

Extracts of commercial potting soils or composted manure from horses or cows were prepared according to this method. Different volumes of soil extract (SEM) were added to 100 ml of CORE1 or CORE2 mediums (see EXAMPLES 15 and 16). For example, SEMf-2 is horse manure extract, SEM is solid extracts from soil samples collected in a greenhouse on various dates, and SEC is liquid cow waste from a dairy farm in Sequim (see EXAMPLE 18 below).

In addition, a commercial organic potting soil mix, BLACK GOLD® potting soil (SUN GRO HORTICULTURE, Bellevue, Wash.), was efficient in augmenting Alga X growth. This soil mix includes 40% to 50% Canadian sphagnum moss, composted softwood bark, rice hulls, pumice, cinders or horticultural grade perlite, worm castings, and continuous release fertilizer. The soil mix contained 0.036% ammonical nitrogen, 0.042% nitrate nitrogen, 0.01% soluble nitrogen, 0.037% insoluble nitrogen, 0.005% urea, 0.05% P₂O₅, and 0.10% potash.

### Example 15

### CORE1 Medium

CORE1 medium is similar to RAC1, without the ALGA-GRO® medium concentrate. The following components below were mixed and the final volume adjusted to 1 L with double-distilled water (ddH₂O).

The following procedure was used to make CORE1 medium:

(1) Thaw frozen f/2 vitamin solution, shake gently.

(2) Add 950 ml ddH₂O to 1000 ml beaker.

(3) Add a stir bar, place on the stir plate and begin stirring.

(4) Using pipettes, measure stock solutions and add them, in the order listed, to the ddH₂O in the beaker, continuing to stir.

(5) Remove the stir bar and add ddH₂O to bring volume up to 1,000 ml.

(6) Return the stir bar and stir until all is combined.

(7) Using the pH meter, place the electrode into the media, continuing to stir.

(8) When the reading is stabilized, drop in HCl with the Pasteur pipet until the pH reads 8.5.

(9) Aliquot required amounts into flasks, close with plugs or buns and cover with paper autoclave bags.

(10) With permanent ink pen, label each bag with type of media, date made and initials of person making it.

(11) Place flasks in autoclave pan.

(12) Proceed with autoclaving 30 minutes at 121° C. on the liquid setting.

| Quantity | Compound | Stock Solution (g/L) | |
|---|---|---|---|
| 1 ml | MgSO₄ 7H₂O | 50 | g/L ddH₂O |
| 1 ml | KCl | 3 | g/L ddH₂O |
| 1 ml | NH₄Cl | 2.68 | g/L ddH₂O |
| 1 ml | NaNO₃ | 20 | g/L ddH₂O |
| 1 ml | beta-glycerophosphate | 2.16 | g/L ddH₂O |
| 1 ml | H₃BO₃ | 0.8 | g/L ddH₂O |
| 1 ml | Na₂EDTA 2H₂O | 8 | g/L ddH₂O |
| 1 ml | FeCl₃ 6H₂O | 1 | g/L ddH₂O |
| 1 ml | CaCl₂ | 75 | g/L ddH₂O |
| 2 ml | NaCl 4M solution | 225.6 | g/L ddH₂O |
| 1 ml | TRIS 3M solution | 363.42 | g/L ddH₂O |

| | | |
|---|---|---|
| 1 ml | DY trace metal ion solution | see above |
| 0.5 ml | f/2 vitamin solution | see above |

### Example 16

### CORE2 Medium

CORE2 medium is similar CORE1 medium, with optimized ingredient quantities and with a different pH buffer, AMPSO. The pH was then adjusted to pH 9.0 and the medium was autoclaved on a liquid cycle. The following components were mixed and the final volume adjusted to 1 L with double-distilled water (ddH₂O) according to the procedure described above in EXAMPLE 15.

| Quantity | Compound | Stock Solution (g/L) |
|---|---|---|
| 1 mL | MgSO₄ 7H₂O | 50 g/L ddH2O |
| 1 mL | KCl | 3 g/L ddH2O |
| 0.5 mL-2 mL | NH₄Cl | 2.68 g/L ddH2O |
| 0.5 mL-2 mL | NaNO₃ | 20 g/L ddH2O |
| 0.33 mL-1 mL | Beta-glycerophosphate | 2.16 g/L ddH2O |
| 1 mL | H₃BO₃ | 0.8 g/L ddH2O |
| 1 mL | Na₂EDTA 2H₂O | 8 g/L ddH2O |
| 1 mL | FeCl₃ 6H₂O | 1 g/L ddH2O |
| 1 mL | CaCl₂ | 75 g/L ddH2O |
| 0 mL-4 mL | NaCl 4 molar solution | 225.6 g/L ddH2O |
| 0.25 mL-16 mL | AMPSO 1.1 molar solution | 250.03 g/L ddH2O |
| 1 mL | DY trace metal ion solution | see above |
| 0.5 mL | f/2 vitamin solution | see above |

### Example 17

### Various Media at Different pH

Six different media were tested for lipid analysis: (1) DYV and ALGA-GRO® (abbreviated “AG”) at a pH 6.8, using MES, initial; (2) DYV and ALGA-GRO® at a pH 6.8, using Tris, initial; (3) RAC1 and ALGA-GRO® at a pH 8.0, using Tris, 40° C., initial; (4) RAC1 and ALGA-GRO® at a pH 8.0, using Tris, tank selected. Notably, the pH may vary from the starting pH as a result of the autoclaving process.

To assess the identity and distribution of fatty acid types both gas chromatography mass spectroscopy (GC/MS) and FAME ionization detection chromatography were used. It was again clear that Alga X cells had numerous fatty acids that were highly unsaturated. The distribution of fatty acids having various chain lengths in Alga X cells grown in different media, and in either small or large volumes, was compared. The data below was gathered from initial and selected cultures. These data suggest that an increase in pH not only increases cell growth rate, but also impacts the distribution of fatty acids in the algal cells.

| Medium | C14 | C16 | C18 | C20 | C22 |
|---|---|---|---|---|---|
| DYV + AG (pH 6.8 MES) initial | 25 | 16 | 49 | 7 | 2 |
| DYV + AG (pH 6.8 Tris) initial | 15 | 21 | 42 | 13 | 9 |
| RAC1 (pH 8.0 Tris) 40 L initial | 10 | 15 | 63 | 6 | 6 |
| RAC1 (pH 8.0 Tris) 1240 L tank | 11 | 17 | 55 | 7 | 6 |
| selected | | | | | |

### Example 18

### Cow Dairy Waste

As described above in EXAMPLE 18, an extract of cow dairy waste (SEC) was added to the control (CORE1+SEM) in the amounts indicated below, the balance being ddH₂O totaling 100 ml. Fatty acids per cell vary only slightly with increasing SEC in the media, increasing with increasing dairy waste on day 3, but decreasing with increasing dairy waste on day 6. Fatty acid profiles remain constant with changing dairy waste extract concentration, with little change in omega 3 and 6 and saturated fatty acid composition.

| | | SEC | | | |
|---|---|---|---|---|---|
| | | 0.25 mL | 0.5 mL | 0.75 mL | 1 mL |
| | | ddH₂O | | | |
| | Day | 99.75 mL | 99.5 mL | 99.25 mL | 99 mL |
| cells/mL | 0 | 1E+05 | 1E+05 | 1E+05 | 1E+05 |
| MC = | 3 | 1.24E+06 | 1.31E+06 | 1.25E+06 | 1.31E+06 |
| 2.11E+06 | 6 | 3.00E+06 | 3.13E+06 | 3.05E+06 | 2.95E+06 |
| | 9 | 4.17E+06 | 4.62E+06 | 4.57E+06 | 4.45E+06 |
| fatty acids/cell | 0 | 2.91E−12 | 2.91E−12 | 2.91E−12 | 2.91E−12 |
| | 3 | 4.13E−12 | 4.20E−12 | 4.22E−12 | 4.29E−12 |
| | 6 | 2.75E−12 | 2.38E−12 | 2.80E−12 | 2.91E−12 |
| fatty acids/g | 9 | 0.165 | 0.142 | 0.136 | 0.114 |
| fatty acids/liter | 0 | 0.0061 | 0.0061 | 0.0061 | 0.0061 |
| | 3 | 0.0051 | 0.0055 | 0.0053 | 0.0056 |
| | 6 | 0.0083 | 0.0074 | 0.0085 | 0.0086 |
| | 9 | X | X | X | X |
| Total w3 + w6 | 0 | 47.1 | 47.1 | 47.1 | 47.1 |
| | 3 | 47.6 | 47.6 | 49.2 | 47.5 |
| | 6 | 39.5 | 42.9 | 43.8 | 39.8 |
| | 9 | 33.5 | 33.4 | 33.2 | 35.9 |
| Saturates | 0 | 43.1 | 43.1 | 43.1 | 43.1 |
| | 3 | 43.5 | 41.1 | 42.2 | 41.4 |
| | 6 | 36.0 | 35.7 | 41.0 | 35.8 |
| | 9 | 29.6 | 33.2 | 28.8 | 27.4 |
| Lipid Profile | 0 | | | | |
| C14s | | 18.0 | 18.0 | 18.0 | 18.0 |
| C16s | | 24.6 | 24.6 | 24.6 | 24.6 |
| C18s | | 29.0 | 29.0 | 29.0 | 29.0 |
| C20+s | | 28.5 | 28.5 | 28.5 | 28.5 |
| | 3 | | | | |
| C14s | | 18.4 | 18.0 | 18.3 | 17.7 |
| C16s | | 22.6 | 21.7 | 22.3 | 22.2 |
| C18s | | 29.1 | 30.4 | 28.4 | 30.2 |
| C20+s | | 30.0 | 29.9 | 31.1 | 30.0 |
| | 6 | | | | |
| C14s | | 16.4 | 16.6 | 17.9 | 16.7 |
| C16s | | 20.9 | 20.1 | 23.0 | 20.6 |
| C18s | | 41.1 | 37.4 | 35.4 | 41.7 |
| C20+s | | 21.6 | 25.8 | 23.8 | 21.1 |
| | 9 | | | | |
| C14s | | 13.2 | 15.0 | 12.4 | 13.3 |
| C16s | | 17.7 | 19.2 | 17.2 | 16.9 |
| C18s | | 53.8 | 54.0 | 60.1 | 55.6 |
| C20+s | | 15.3 | 11.8 | 10.3 | 14.2 |

### Example 19

### 100% Clear Waste Water

One liter of 100% Clear waste water (human) was added to 100 ml ddH2O was compared against one liter of 100% Clear waste water (human) plus 100 ml SEM and the control (CORE1+SEM). The samples were grown under a light intensity of 100 ue/m2/sec+/−10 at about 21° C. on a 12 L/12 D light cycle. Fatty acids per gram dry weight marginally higher than control (CORE1+SEM). Fatty acid profiles remain very similar.

| Clear | | 1 L | 1 L | |
|---|---|---|---|---|
| Other | Day | 100 ml ddH2O | 100 ml SEM | Control |
| cells/mL | 9 | 2.50E+06 | 2.71E+06 | 3.00E+06 |
| fatty acids/cell | 9 | X | X | X |
| fatty acids/gram | 9 | 0.0694 | 0.0551 | 0.0505 |
| dry weight | | | | |
| Total w3 + w6 | 9 | 29.2 | 28.9 | 32.3 |
| Saturates | 9 | 51.7 | 51.3 | 52.7 |
| Lipid Profile | | | | |
| C14s | 9 | 21.8 | 21.9 | 22.3 |
| C16s | | 27.1 | 26.6 | 27.5 |
| C18s | | 39.5 | 40.6 | 34.8 |
| C20+s | | 11.6 | 10.9 | 15.4 |

### Example 20

### Clear Wastewater

One liter of 100% Clear waste water (human) was added to various mixtures of CORE1, ddH2O, and SEM, and compared against the control (CORE1+SEM). The samples were grown under a light intensity of 100 ue/m2/sec+/−10 at about 21° C. on a 12 L/12 D light cycle. Limiting CORE increases lipid per cell by upwards of 75%. Fatty acid profiles, however, remain largely unaffected by this increase, though a slight increase in saturated fatty acids is observed.

| | | Clear | | | | |
|---|---|---|---|---|---|---|
| | | 1 L | 1 L | 1 L | 1 L | |
| | | CORE1 | | | | |
| | | ~7 ml | ~7 ml | ~14 ml | ~14 ml | |
| | | Other | | | | |
| | Day | 100 ml H2O | 100 ml SEM | 100 ml H2O | 100 ml SEM | Control |
| cells/mL | 0 | 1.0E+05 | 1.0E+05 | 1.0E+05 | 1.0E+05 | 1.0E+05 |
| MC = | 3 | 1.17E+06 | 1.18E+06 | 1.20E+06 | 1.28E+06 | 1.08E+06 |
| 2.91E+06 | 6 | 4.83E+06 | 4.91E+06 | 4.43E+06 | 5.46E+06 | 2.70E+06 |
| | 9 | 4.98E+06 | 5.34E+06 | 2.75E+06 | 7.16E+06 | 3.90E+06 |
| fatty | 0 | | 0.00E+00 | 0.00E+00 | 0.00E+00 | 0.00E+00 |
| acids/cell | 3 | 1.14E−11 | 1.23E−11 | 1.20E−11 | 1.05E−11 | 1.20E−11 |
| (g) | 6 | 7.07E−12 | 6.47E−12 | 3.64E−12 | 4.80E−12 | 4.65E−12 |
| fatty acids/g | 9 | 0.185 | 0.164 | 0.153 | 0.209 | |
| dry weight | | | | | | |
| fatty | 0 | 0.0000 | 0.0000 | 0.0000 | 0.0000 | 0.0000 |
| acids/liter | 3 | 0.0133 | 0.0145 | 0.0144 | 0.0134 | 0.0130 |
| (g) | 6 | 0.0341 | 0.0318 | 0.0161 | 0.0262 | 0.0126 |
| | 9 | X | X | X | X | |
| Total w3 + | 0 | | 0.0 | 0.0 | 0.0 | 0.0 |
| w6 | 3 | 35.0 | 35.8 | 36.5 | 37.9 | 37.4 |
| (%) | 6 | 33.3 | 34.4 | 32.0 | 34.4 | 36.2 |
| | 9 | 31.3 | 30.9 | 27.0 | 31.7 | |
| Saturates | 0 | | 0.0 | 0.0 | 0.0 | 0.0 |
| (%) | 3 | 33.7 | 31.9 | 33.4 | 33.3 | 31.7 |
| | 6 | 35.7 | 37.0 | 32.5 | 29.1 | 27.4 |
| | 9 | 43.2 | 42.3 | 34.0 | 38.0 | |
| Lipid Profile | | | | | | |
| C14s | 0 | x | x | x | x | x |
| C16s | | x | x | x | x | x |
| C18s | | x | x | x | x | x |
| C20+s | | x | x | x | x | x |
| C14s | 3 | 13.4 | 12.9 | 13.8 | 13.4 | 13.1 |
| C16s | | 18.9 | 18.2 | 18.9 | 18.8 | 17.4 |
| C18s | | 56.9 | 57.3 | 54.4 | 54.9 | 59.2 |
| C20+s | | 10.9 | 11.7 | 12.9 | 13.0 | 10.3 |
| C14s | 6 | 14.7 | 15.0 | 14.4 | 12.8 | 12.2 |
| C16s | | 19.5 | 19.8 | 19.8 | 17.0 | 15.9 |
| C18s | | 56.0 | 54.8 | 56.0 | 58.9 | 57.8 |
| C20+s | | 9.8 | 10.5 | 9.8 | 11.5 | 14.1 |
| C14s | 9 | 18.9 | 18.5 | 14.6 | 17.8 | |
| C16s | | 22.4 | 22.1 | 18.5 | 19.7 | |
| C18s | | 48.6 | 49.3 | 55.3 | 52.1 | |
| C20+s | | 10.1 | 10.1 | 11.6 | 10.4 | 100.0 |

### Example 21

### UV Wastewater

One liter of 100% UV waste water (human) was added to a mixture of CORE1 and SEM, and compared against the control (CORE1+SEM). The samples were grown under a light intensity of 100 ue/m2/sec+/−10 at about 21° C. on a 12 L/12 D light cycle. The addition of UV-treated wastewater to the control medium (CORE1+SEM) caused a slight increase in lipid per cell over control. Fatty acid profiles remain unaffected in any significant way.

| | | 100 ml SEM | Control |
|---|---|---|---|
| | | 1 L UV WW | (CORE1 + |
| | Day | 14 ml CORE1 | SEM) |
| cells/mL | 0 | 1.0E+05 | 1.0E+05 |
| MC = | 3 | 1.17E+06 | 1.38E+06 |
| 2.68E+06 | 6 | 5.94E+06 | 3.48E+06 |
| | 9 | 3.52E+06 | 4.88E+06 |
| fatty | 0 | 1.82E−12 | 1.82E−12 |
| acids/cell | 3 | 5.82E−12 | 4.76E−12 |
| | 6 | 2.36E−12 | 2.53E−12 |
| | 9 | X | X |
| fatty | 0 | 0.0049 | 0.0049 |
| acids/liter | 3 | 0.0068 | 0.0066 |
| | 6 | 0.0140 | 0.0088 |
| | 9 | | |
| Total w3 + | 0 | 47.3 | 47.3 |
| w6 | 3 | 33.8 | 35.4 |
| | 6 | 31.0 | 35.3 |
| | 9 | | |
| Saturates | 0 | 39.5 | 39.5 |
| | 3 | 46.9 | 48.8 |
| | 6 | 42.2 | 35.6 |
| | 9 | | |
| Lipid Profile | 0 | | |
| C14s | | 18.9 | 18.9 |
| C16s | | 22.2 | 22.2 |
| C18s | | 29.5 | 29.5 |
| C20+s | | 29.4 | 29.4 |
| | 3 | | |
| C14s | | 18.9 | 19.0 |
| C16s | | 25.4 | 25.9 |
| C18s | | 37.9 | 36.8 |
| C20+s | | 17.8 | 18.3 |
| | 6 | | |
| C14s | | 20.1 | 16.4 |
| C16s | | 22.2 | 21.2 |
| C18s | | 44.7 | 43.7 |
| C20+s | | 13.0 | 18.7 |

### Example 22

### Fatty Acid Content Over Time

Alga X was cultured in a standard medium (CORE1+SEM) with a photoperiod of 12 hours of light followed by 12 hours of dark. Alga cells were collected for fatty acid analysis at 0, 3, 4, and 8 days. The resultant data provided below shows that an Alga X culture has very little change in fatty acid chain length distribution over time. Notably, the highest percentage of saturated fatty acids compared to unsaturated fatty acids is achieved on day 4. Saturated fatty acid percentage is important because saturated fatty acids are more commonly used in biodiesel applications than unsaturated fatty acids. However, both saturated and unsaturated fatty acids may be used for biodiesel applications.

| | |
|---|---|
| | % Sat. to |
| Percent of Total Fatty Acids | Unsat. |

| Day | C14 | C16 | C18 | C20 | Fatty Acids |
|---|---|---|---|---|---|
| day 0 | 12.7 | 26.3 | 45.2 | 15.8 | 33.1 |
| day 3 | 15.7 | 22.3 | 48.3 | 13.7 | 37.5 |
| day 4 | 16.8 | 21.4 | 48.0 | 13.8 | 40.0 |
| day 8 | 15.9 | 21.7 | 46.4 | 16.6 | 37.1 |

### Example 23

### Acetate

The samples were grown under a light intensity of 100 ue/m2/sec+/−10 at about 21° C. on a 12 L/12 D light cycle. Increased acetate in the medium caused a slight increase in total fatty acids, particularly in the exponential phase, corrected for cell count this leads to an overall increase in productivity. Omega 3 and 6 fatty acids are slightly suppressed with increased acetate, while saturated fats remain unchanged. Lipid profiles similar across all concentrations.

| Acetate | Day | 100 uM | 200 uM | 400 uM | Control |
|---|---|---|---|---|---|
| cells/mL | 0 | 1.0E+05 | 1.0E+05 | 1.0E+05 | 1.0E+05 |
| MC = | 3 | 1.54E+06 | 1.69E+06 | 1.67E+06 | 1.79E+06 |
| 2.96E+06 | 6 | 3.27E+06 | 4.02E+06 | 4.44E+06 | 3.08E+06 |
| | 9 | 4.87E+06 | 5.02E+06 | 4.70E+06 | 4.77E+06 |
| fatty | 0 | 2.79E−12 | 2.79E−12 | 2.79E−12 | 2.79E−12 |
| acids/cell | 3 | 2.92E−12 | 2.85E−12 | 3.28E−12 | 2.33E−12 |
| (g) | 6 | 2.19E−12 | 2.57E−12 | 2.56E−12 | 2.31E−12 |
| fatty acids/g | 9 | | | | |
| dry weight | | | | | |
| fatty | 0 | 0.0082 | 0.0082 | 0.0082 | 0.0082 |
| acids/liter | 3 | 0.0045 | 0.0048 | 0.0055 | 0.0042 |
| (g) | 6 | 0.0072 | 0.0103 | 0.0114 | 0.0071 |
| | 9 | X | X | X | |
| Total w3 + | 0 | 37.4 | 37.4 | 37.4 | 37.4 |
| w6 | 3 | 45.0 | 45.1 | 41.4 | 52.0 |
| (%) | 6 | 37.0 | 33.0 | 34.7 | 38.7 |
| | 9 | | | | |
| Saturates | 0 | 26.9 | 26.9 | 26.9 | 26.9 |
| (%) | 3 | 34.5 | 35.2 | 35.0 | 34.8 |
| | 6 | 27.7 | 29.4 | 36.0 | 27.5 |
| | 9 | | | | |
| Lipid Profile | | | | | |
| C14s | 0 | 13.0 | 13.0 | 13.0 | 13.0 |
| C16s | | 16.0 | 16.0 | 16.0 | 16.0 |
| C18s | | 52.4 | 52.4 | 52.4 | 52.4 |
| C20+s | | 18.6 | 18.6 | 18.6 | 18.6 |
| C14s | 3 | 15.0 | 15.3 | 14.9 | 15.9 |
| C16s | | 19.4 | 20.2 | 21.3 | 18.3 |
| C18s | | 40.7 | 39.8 | 41.4 | 38.0 |
| C20+s | | 24.9 | 24.7 | 22.4 | 27.8 |
| C14s | 6 | 12.3 | 12.8 | 16.5 | 12.7 |
| C16s | | 17.5 | 17.7 | 20.6 | 17.1 |
| C18s | | 50.9 | 53.9 | 48.0 | 50.3 |
| C20+s | | 19.3 | 15.6 | 14.9 | 19.9 |
| C14s | 9 | | | | |
| C16s | | | | | |
| C18s | | | | | |
| C20+s | | 100.0 | 100.0 | 100.0 | 100.0 |

### Example 24

### Semi-Continuous Culture Experiment

The samples were grown under a light intensity of 100 ue/m2/sec+/−10 on a 12 L/12 D light cycle at 20° C. in a 5 liter semi-continuous batch (or “bump”) culture. The cultures were grown on the control medium (CORE1+SEM). The bump culture is compared in the table below against a continuous culture and a control culture (which have the same continuous conditions, except that they start at different culture densities).

Fatty acids per cell stable after bump, with lipid profiles of post-bump samples closely resembling those of day 6 controls. Control day 9s in this experiment have a high fatty acid per cell content, likely because the cells have not divided. A plot of another semi-continuous batch (or “bump”) culture experiment are shown in FIG. 7.

| | Day | Semi-Cont. | Continuous | Control |
|---|---|---|---|---|
| cells/mL | 0 | 7.87E+05 | 7.87E+05 | 1.0E+05 |
| MC = | 3 | 2.60E+06 | 2.61E+06 | 1.32E+06 |
| 3.70E+06 | 5 | 2.41E+06 | 3.59E+06 | 2.48E+06 |
| | 6 | 2.69E+06 | 4.05E+06 | 2.79E+06 |
| | 9 | 3.63E+06 | 3.72E+06 | 3.51E+06 |
| | 10 | 3.22E+06 | X | X |
| | 12 | 3.05E+06 | X | X |
| | 14 | 3.08E+06 | X | X |
| | 15 | 3.06E+06 | X | X |
| fatty acids/cell | 0 | 2.25E−12 | 2.25E−12 | 2.25E−12 |
| (g) | 3 | 2.38E−12 | 2.24E−12 | 3.33E−12 |
| | 5 | 1.76E−12 | 2.27E−12 | 2.00E−12 |
| | 6 | 2.54E−12 | 2.23E−12 | 2.45E−12 |
| | 9 | 2.60E−12 | 4.74E−12 | 4.31E−12 |
| | 10 | 3.19E−12 | | |
| | 12 | 2.68E−12 | | |
| | 14 | 3.56E−12 | | |
| | 15 | 2.71E−12 | | |
| fatty acids/liter | 0 | 0.0083 | 0.0083 | 0.0083 |
| (g) | 3 | 0.0062 | 0.0058 | 0.0044 |
| | 5 | 0.0042 | 0.0081 | 0.0050 |
| | 6 | 0.0068 | 0.0090 | 0.0068 |
| | 9 | 0.0094 | 0.0176 | 0.0151 |
| | 10 | 0.0103 | X | X |
| | 12 | 0.0082 | X | X |
| | 14 | 0.0110 | X | X |
| | 15 | 0.0083 | X | X |
| Total w3 + w6 | 0 | 36.4 | 36.4 | 36.4 |
| (%) | 3 | 36.5 | 41.8 | 42.5 |
| | 5 | 56.6 | 37.2 | 48.1 |
| | 6 | 41.6 | 34.8 | 41.4 |
| | 9 | 34.2 | 30.2 | 32.4 |
| | 10 | 34.9 | X | X |
| | 12 | 36.8 | X | X |
| | 14 | 37.3 | X | X |
| | 15 | 35.5 | X | X |
| Saturates | 0 | 28.3 | 28.3 | 28.3 |
| (%) | 3 | 27.9 | 28.5 | 38.3 |
| | 5 | 35.2 | 23.8 | 28.3 |
| | 6 | 28.7 | 25.1 | 27.2 |
| | 9 | 26.6 | 33.5 | 30.5 |
| | 10 | 30.2 | X | X |
| | 12 | 27.6 | X | X |
| | 14 | 27.1 | X | X |
| | 15 | 31.0 | X | X |
| Lipid Profile | | | | |
| C14s | 0 | 14.3 | 14.3 | 14.3 |
| C16s | | 15.8 | 15.8 | 15.8 |
| C18s | | 50.1 | 50.1 | 50.1 |
| C20+s | | 19.8 | 19.8 | 19.8 |
| C14s | 3 | 12.9 | 13.5 | 17.5 |
| C16s | | 16.1 | 16.5 | 19.3 |
| C18s | | 44.7 | 46.8 | 39.7 |
| C20+s | | 26.3 | 23.2 | 23.5 |
| C14s | 5 | 17.4 | 11.0 | 14.2 |
| C16s | | 21.4 | 14.9 | 15.3 |
| C18s | | 22.6 | 50.5 | 38.9 |
| C20+s | | 38.6 | 23.6 | 31.6 |
| C14s | 6 | 13.4 | 11.4 | 13.3 |
| C16s | | 17.2 | 15.7 | 15.5 |
| C18s | | 47.1 | 54.2 | 47.6 |
| C20+s | | 22.3 | 18.7 | 23.6 |
| C14s | 9 | 12.4 | 13.6 | 13.0 |
| C16s | | 16.4 | 20.5 | 18.4 |
| C18s | | 54.9 | 52.8 | 56.8 |
| C20+s | | 16.3 | 13.1 | 11.8 |
| C14s | 10 | 13.1 | X | X |
| C16s | | 18.0 | X | X |
| C18s | | 53.1 | X | X |
| C20+s | | 15.8 | X | X |
| C14s | 12 | 12.7 | X | X |
| C16s | | 16.8 | X | X |
| C18s | | 52.1 | X | X |
| C20+s | | 18.4 | X | X |
| C14s | 14 | 12.7 | X | X |
| C16s | | 16.3 | X | X |
| C18s | | 52.5 | X | X |
| C20+s | | 18.5 | X | X |
| C14s | 15 | 13.8 | X | X |
| C16s | | 18.7 | X | X |
| C18s | | 52.2 | X | X |
| C20+s | | 15.3 | X | X |

### Example 25

### Phosphate

Phosphate is an important food source for algal cultures and an expensive ingredient. Attempts were made to determine if the amount of phosphate in the CORE1 medium is already saturated, and if the amount of phosphate in the medium can be decreased, but still resulting in good growth rates.

This experiment showed that phosphate is needed for the survival of the cells. The data from the flask without any B-glycerolphosphate added show that the cells failed to continue to increase in number after day 4. The culture with ⅓ and ⅔ the amount of B-glycerolphosphate grew the same or slightly better than the control (6.45 uM). This data shows that phosphate can be decreased by ⅔ and still enable good cell growth.

| BGlycerol- | | | | | |
|---|---|---|---|---|---|
| phosphate | Day | 0 | 2.13 uM | 4.32 uM | 6.45 uM |
| cells/mL | 0 | 1.0E+05 | 1.0E+05 | 1.0E+05 | 1.0E+05 |
| MC = | 2 | 6.00E+05 | 6.62E+05 | 7.16E+05 | 6.98E+05 |
| 2.58E+06 | 3 | 1.29E+06 | 1.46E+06 | 1.58E+06 | 1.57E+06 |
| | 4 | 1.86E+06 | 2.72E+06 | 2.81E+06 | 2.75E+06 |
| | 6 | 1.92E+06 | 3.99E+06 | 3.79E+06 | 3.54E+06 |
| | 8 | 1.94E+06 | 4.70E+06 | 4.78E+06 | 4.10E+06 |
| | 9 | 2.01E+06 | 4.98E+06 | 4.90E+06 | 4.53E+06 |

In another experiment, with varying phosphate levels in the medium, fatty acid productivity increases slightly with increasing KH₂PO₄ by day 6, though the increase is small and may or may not be significant. Fatty acid profiles remain fairly constant with changing phosphate levels.

### Example 26

### Nitrogen

As shown by the data below, lipid per cell is little affected by increased nitrogen in the media, though it may be slightly suppressed by very high levels of nitrates.

Fatty Acid Profile: High nitrate levels increase omega 3 and 6 fatty acid content, though saturated fatty acid content remains unaffected. C18s suppressed by high nitrate concentration. Cells appear acclimated by day 6.

| | | | | 2xNO3 | | |
|---|---|---|---|---|---|---|
| | Day | 2xNO3 | 2xNH4 | 2xNH4 | 6xNO3 | Control |
| cells/mL | 0 | | | | | |
| | 3 | 1.27E+06 | 1.43E+06 | 1.44E+06 | 1.07E+06 | 1.28E+06 |
| | 6 | 3.01E+06 | 2.82E+06 | 2.88E+06 | 2.35E+06 | 2.94E+06 |
| | 9 | 4.05E+06 | 3.27E+06 | 3.87E+06 | 3.43E+06 | 3.80E+06 |
| fatty acids/cell | 0 | | | | | |
| | 3 | 3.91E−12 | 3.60E−12 | 3.24E−12 | 3.46E−12 | 3.97E−12 |
| | 6 | 2.27E−12 | 2.40E−12 | 2.49E−12 | 2.48E−12 | 2.23E−12 |
| fatty acids/g | 9 | 0.141 | 0.114 | 0.112 | 0.0944 | 0.108 |
| fatty | 0 | 0.0000 | 0.0000 | 0.0000 | 0.0000 | 0.0000 |
| acids/liter | 3 | 0.0050 | 0.0051 | 0.0047 | 0.0037 | 0.0051 |
| | 6 | 0.0068 | 0.0068 | 0.0072 | 0.0058 | 0.0065 |
| | 9 | X | X | X | X | X |
| Total w3 + w6 | 0 | | | | | |
| | 3 | 41.7 | 43.8 | 42.9 | 47.1 | 40.8 |
| | 6 | 39.5 | 38.9 | 38.0 | 37.1 | 39.6 |
| | 9 | 36.9 | 35.2 | 34.8 | 37.2 | 34.2 |
| Saturates | 0 | | | | | |
| | 3 | 43.9 | 42.8 | 46.7 | 44.1 | 45.1 |
| | 6 | 34.1 | 34.3 | 33.2 | 34.7 | 34.1 |
| | 9 | 28.7 | 29.4 | 25.1 | 28.5 | 25.3 |
| Lipid Profile | 0 | | | | | |
| C14s | | X | X | X | X | X |
| C16s | | X | X | X | X | X |
| C18s | | X | X | X | X | X |
| C20+s | | X | X | X | X | X |
| C14s | 3 | 18.2 | 18.1 | 19.8 | 18.0 | 18.7 |
| C16s | | 24.4 | 24.3 | 25.6 | 25.2 | 25.2 |
| C18s | | 36.7 | 34.3 | 26.8 | 29.5 | 35.5 |
| C20+s | | 20.8 | 23.3 | 28.0 | 27.4 | 20.7 |
| C14s | 6 | 15.3 | 15.3 | 15.3 | 16.0 | 15.6 |
| C16s | | 20.8 | 21.3 | 20.3 | 21.3 | 20.4 |
| C18s | | 41.7 | 42.1 | 44.9 | 43.6 | 43.2 |
| C20+s | | 22.4 | 21.5 | 19.6 | 19.2 | 20.8 |
| C14s | 9 | 13.6 | 13.3 | 11.5 | 13.7 | 11.4 |
| C16s | | 17.8 | 18.9 | 16.7 | 18.1 | 16.7 |
| C18s | | 52.9 | 53.9 | 59.4 | 54.5 | 59.6 |
| C20+s | | 15.7 | 13.9 | 12.4 | 13.7 | 12.3 |

### Example 27

### Media Comparison

The starting culture densities were 5.00E+03 cells/ml. The table below shows comparative data for DYV, RAC1, CORE1+horse manure soil extract, and CORE1+an organic soil extract (e.g., BLACK GOLD® potting soil in EXAMPLE 14 above). The data shows that CORE1+soil extract provided a significant culture density increase over DYV and RAC1.

| Media | 6 Days | 8 Days |
|---|---|---|
| DYV | 3.69E+04 | 7.38E+04 |
| RAC1 | 7.00E+05 | 1.62E+06 |
| CORE1 + Horse | 1.37E+06 | 2.66E+06 |
| CORE1 + Organic | 7.63E+05 | 2.32E+06 |

### Example 28

### Media Comparison

The starting culture densities were 1.00E+05 cells/ml. The table below shows comparative data for CORE1+SEM (soil extract), CORE1+SEC (cow dairy waste extract), and CORE1+Clear (human waste water)+SEM. The data shows that CORE1+Clear (human waste water human)+SEM provided a significant culture density increase over CORE1+SEM (soil extract), CORE1+SEC (cow dairy waste extract).

| Media | 6 Days (×10 E6) | 8 Days (×10 E6) |
|---|---|---|
| CORE1 + SEM | 2.79 | 4.17 |
| | 3.21 | 4.37 |
| | 3.01 | 4.05 |
| | 3.18 | 4.65 |
| | 3.19 | 4.70 |
| CORE1 + SEC | 2.81 | 3.69 |
| | 2.95 | 4.25 |
| CORE1 + Clear + | 5.46 | 7.16 |
| SEM | | |
| | 5.66 | 7.17 |

### Example 29

### Media Comparison

Below is a comparison chart for fatty acid profiles for Alga X grown in different media. In general, CORE1+SEM+Clear media produces the best results in terms of percentage of saturates.

| | | | | CORE1 + SEM + |
|---|---|---|---|---|
| | DYV | DYV + AG | CORE1 + SEM | Clear |
| Total w3 + w6 | 53.5 | 19.6 | 27.4 | 30.9 |
| Saturates(%) | 34.0 | 14.8 | 36.2 | 42.3 |
| Lipid Profile | | | | |
| C14s | 22.0 | 5.5 | 12.2 | 18.5 |
| C16s | 18.5 | 13.8 | 15.9 | 22.1 |
| C18s | 46.5 | 61.2 | 57.8 | 49.3 |
| C20+s | 9.0 | 28.1 | 14.1 | 10.1 |

### Example 30

### BODIPY 505/515

A 5 mM stock solution of BODIPY 505/515 (4,4-difluoro-1,3,5,7-tetramethyl-4-bora-3a,4a-diaza-s-indacene, MW 248) was made by dissolving the dye in anhydrous dimethyl sulfoxide (DMSO). 10 micro-liters of a 5 mM BODIPY 505/515 DMSO solution was diluted into 50 ml of algal suspension. The final labeling solution contained 1 micro-molar BODIPY 505/515 and 0.05% DMSO. Within 5 minutes after dye addition, fluorescently-labeled lipid bodies in algal cells are visible using either an epifluorescence microscope (FIG. 8) or a confocal microscope. The images in FIG. 8 were taken on a Zeiss Meta Scanning Confocal Light Microscope at the University of Washington Center for Nanotechnology User Facility.

### Example 31

### Cell Sorting

The first sort was accomplished using cells stained with Nile red dye and subsequent selections were done with BODIPY 505/515 dye-stained cells. After each sorting, cells were grown in appropriate medium (either RAC1 or CORE1+SEM) until robust cultures were generated. Cells were maintained in a number of different culture vessels after sorting: in 48 or 96 well plates (approximately 100 to 500 ul); small 50 ml falcon culture flasks (10 ml); 250 ml Erlenmeyer flasks (100 ml) or 2,800 ml fernbach flasks (1000 ml). Medium used for culturing was either RAC1 or CORE1+SEM. Light intensity was from 40 to 100 ue/m²sec and temperature was 20° C. Length of culture maintenance was dependent on the final desired culture density.

Following three sortings, the cells were tested for relative lipid content using both flow cytometry and mass spectroscopy. A culture that had never been sorted was used as the non-selected control. Though both experimental and control cultures were in logarithmic growth phase, (selected: 1.65+/−0.03×10⁶ cells/ml; non-selected 2.16+/−0.04×10⁶ cells/ml), the relative lipid dye signal (FU=fluorescent units) measured after flow-cytometric analysis for the selected (354.4+/−44.3 FU) and non-selected (141.6+/−6.1 FU) populations differed. Subtracted background was 3.14 FU. This change in lipid content per cell was verified by mass spectroscopic analysis of lipids extracted from these same experimental cultures. Selected cells contained 3.21+/−0.06×10⁻¹² and non-selected cells had 2.81+/−0.02×10⁻¹² g fatty acids/cell respectively (14% difference). As these experimental cultures aged, the signal between non-selected and selected cell populations was also evident. Selected cells (3.27+/−0.06×10⁶ cells/ml) and non-selected (3.88+/−0.04×10⁶ cells/ml) had mean fluorometric signals of (202.2+/−5.7FU) and (144.6+/−30.1 FU) respectively. Subtracted background was 3.14 FU.

### Example 32

### Agitation

Alga X cell cultures were subjected to agitation between 0 and 100 rpm. Below is a comparison table for cell growth at various agitation speeds: control (0 rpm), 30 rpm, 60 rpm, 80 rpm, and 100 rpm. The results show that cell growth at 0 rpm, 30 rpm, and 60 rpm had comparable cell growth after 12 days, with the culture at 60 rpm agitation speed reaching an early peak of 4.63E+06 on day 7. By comparison, the cultures subjected to no agitation and about 30 rpm agitation reached similar cell concentrations on day 9. The cultures agitated at 80 rpm and 100 rpm did not produce comparable cell growth to the cultures agitated at lower agitations speeds or at no agitation. Therefore, the 80 rpm and 100 rpm experiments were canceled after day 4.

All cell cultures were grown at a light intensity of 85+/−15 ue/m²/sec on a 12 L/12 D light cycle at about 21° C. in a 1100 ml flask. The original stock culture was maintained in a medium of distilled water+CORE1+SEM (soil extract), with a concentration of about 2.77E+06 cells/ml. A volume of about 39.7 ml of original stock was added to each starting flask, with the balance being distilled water+CORE1+SEM to bring the cell cultures to a concentration of about 1.00E+06.

| | Cell Culture Density (cells/ml) based on | | | | |
|---|---|---|---|---|---|
| | Agitation Speed (rpm) | | | | |
| Day | 0 | 30 | 60 | 80 | 100 |
| day 0 | 1.00E+05 | 1.00E+05 | 1.00E+05 | 1.00E+05 | 1.00E+05 |
| day 2 | 6.05E+05 | 6.82E+05 | 6.66E+05 | 3.13E+05 | 2.31E+05 |
| day 4 | x | x | x | 1.30E+06 | 8.58E+05 |
| day 5 | 2.80E+06 | 2.92E+06 | 3.60E+06 | x | x |
| day 7 | 3.91E+06 | 3.85E+06 | 4.63E+06 | x | x |
| day 9 | 4.73E+06 | 4.55E+06 | 4.52E+06 | x | x |
| day 12 | 4.79E+06 | 4.63E+06 | 4.31E+06 | x | x |

### Example 33

### Sodium Bicarbonate

All cell cultures were grown at a light intensity of 85+/−15 μe/m²/sec on a 12 L/12 D light cycle at about 21° C. in a 1100 ml flask. The original stock culture was maintained in a medium of distilled water+CORE1+SEM, with a concentration of about 2.92E+06 cells/ml. A volume of about 37.6 ml of original stock was added to each starting flask, with the balance being medium to bring the cell cultures to a concentration of about 1.00E+06.

The Control-1 culture was maintained in a medium of CORE1, SEM, with TRIS buffer solution. The Control-2 culture was maintained in a medium of CORE1, SEM, with 16 mM AMPSO pH buffer solution. The AMPSO-1 culture was maintained in a medium of CORE1, SEM, 8 mM AMPSO pH buffer solution, and 2 mM NaHCO₃. The AMPSO-2 culture was maintained in a medium of CORE1, SEM, 8 mM AMPSO pH buffer solution, and 4 mM NaHCO₃. The AMPSO-3 culture was maintained in a medium of CORE1, SEM, 8 mM AMPSO pH buffer solution, and 6 mM NaHCO3.

By comparing the culture growth of Control-1 and Control-2, the results show that buffering pH at more alkaline conditions, for example, with AMPSO pH buffer (which buffers pH in an alkaline range of about 8.3 to about 10), instead of TRIS pH buffer (which buffers pH in an alkaline range of about 7 to about 9), results in improved cell culture growth. The results further show that sodium bicarbonate (with AMPSO pH buffer) further enhances cell culture growth, whether in 2 mM, 4 mM, or 6 mM amounts.

| | Cell Culture Density (cells/ml) | | | | |
|---|---|---|---|---|---|
| | | | AMPSO-1 + | AMPSO-2 + | AMPSO-3 + |
| | Control-1 + | | 2 mM | 4 mM | 6 mM |
| Day | TRIS | Control-2 + AMPSO | NaHCO3 | NaHCO3 | NaHCO3 |
| day 0 | 1.00E+05, | 1.00E+05, | 1.00E+05, | 1.00E+05, | 1.00E+05, |
| | pH ~8.00 | pH 9.00 | pH 9.00 | pH 9.00 | pH 9.00 |
| day 2 | 5.68E+05, | 3.82E+05, | 3.61E+05, | 3.60E+05, | 3.24E+05, |
| | pH 8.24 | pH 8.96 | pH 8.98 | pH 8.99 | pH 9.01 |
| day 5 | 2.59E+06, | 3.23E+06, | 5.00E+06, | 4.53E+06, | 4.33E+06, |
| | pH 8.60 | pH 9.02 | pH 9.18 | pH 9.19 | pH 9.24 |
| day 6 | 3.02E+06, | 4.07E+06, | 5.95E+06, | 5.75E+06, | 5.58E+06, |
| | pH 8.60 | pH 8.98 | pH 9.16 | pH 9.16 | pH 9.21 |
| day 7 | 3.87E+06, | 4.69E+06, | 5.90E+06, | 5.97E+06, | 5.75E+06, |
| | pH 8.46 | pH 8.94 | pH 9.07 | pH 9.07 | pH 9.08 |

### Example 34

### Sodium Bicarbonate and Waste Water

All cell cultures were grown at a light intensity of 85+/−15 μe/m²/sec on a 12 L/12 D light cycle at 21° C. in a 1100 ml flask. The original stock culture was maintained in CORE1 (distilled water based medium), plus SEM, with a concentration of about 3.19E+06 cells/ml. Volumes of 34.5 ml of original stock were added to each starting flask, with the balance being medium to bring the cell cultures to a concentration of about 1.00E+05.

The experimental AMPSO culture was maintained in a medium of Clear (human waste water), SEM, and 8 mM AMPSO pH buffer. The AMPSO plus NaHCO₃ culture was maintained in a medium of Clear (human waste water), SEM, 8 mM AMPSO pH buffer, and 2 mM NaHCO3.

Results show the use of waste water together with AMPSO pH buffer and sodium bicarbonate results in considerably enhanced culture growth, particularly when compared to culture growth in the distilled water CORE1 medium containing AMPSO and sodium bicarbonate described above in EXAMPLE 33.

Cell Culture (cells/ml)

| | | AMPSO + |
|---|---|---|
| Day | AMPSO | NaHCO₃ |
| day 0 | 1.00E+05, | 1.00E+05, |
| | pH ~9.00 | pH ~9.00 |
| day 1 | 1.85E+05, | 1.60E+05, |
| | pH 9.16 | pH 9.15 |
| day 3 | 1.39E+06, | 1.73E+06, |
| | pH 9.31 | pH 9.29 |
| day 6 | 6.61E+06, | 7.31E+06, |
| | pH 9.67 | pH 9.67 |
| day 7 | 7.24E+06 | 7.46E+06 |
| day 8 | 7.19E+06, | 6.78E+06, |
| | pH 9.55 | pH 9.49 |

### Example 35

### Long-Term Semi-Continuous Culture Experiment

The two samples were grown under substantially similar conditions at a light intensity of 100+/−10μe/m²/sec on a 12 L/12 D light cycle at about 21° C. in a 1.1 liter semi-continuous batch (or “bump”) culture. The original stock cultures were maintained in a medium of distilled water+CORE1+SEM. One aliquot of each stock was transferred to a new medium of CLEAR Waste Water 1-4+CORE1+SEM. Every 2-3 days, 50% of the culture volume was removed from each experimental flask when the density was in the range of about 4.5 to about 6.0E+06 cells/mL and sufficient fresh media was added to result in a cell density of about 2.0 to about 3.0E+06 cells/mL. Data for the semi-continuous batch culture growth can be seen in FIG. 9. Every 7 days, 75% of the culture volume was removed from each experimental flask, which accounts for the low dips in the data.

Other semi-continuous batch (or “bump”) culture experiments are described with reference to EXAMPLE 24 or FIG. 7.

### Example 36

### Directed Evolution

Alga X cells were passed through six flow cytometric selections in duplicate (Sorted Alpha and Sorted Beta). At each selection, a 100 ml culture was initiated with 5,000 cells representing the 0.5% of the population having the highest neutral lipid content. Experimental cultures of 1.1 liters were generated for lipid analysis over a growth time. All cultures were maintained at 100 ue/m²/sec with no agitation. Samples were taken at the sixth hour of light in a 12 hour light, 12 hour dark cell cycle.

Referring to FIG. 10 and the data table below, the use of flow cytometry for “directed evolution” produces cultures with a higher fatty acid content. The data in the table below shows that Sorted Alpha and Sorted Beta have significantly higher percentages of saturated fatty acids than the non-sorted control at both day 3 and day 6 in the growth cycle. In that regard, the sorted cells contain saturated fatty acids that are in the range of about 20-25% higher than the non-sorted saturates on day 3 and in the range of about 15-20% higher than the non-sorted saturates on day 6.

| | Control | | |
|---|---|---|---|
| Saturates(%) | (Non-Sorted) | Sorted Alpha | Sorted Beta |
| Day 3 | 42.2 | 50.8 | 52.8 |
| Day 6 | 33.4 | 39.2 | 38.6 |

The data in FIG. 10 shows that as culture cell density increases, the fatty acid per cell of Alga X decreases. However, the Sorted Alpha and Sorted Beta culture still have higher fatty acid content per cell than the control at various cell densities.

While illustrative embodiments have been illustrated and described, it will be appreciated that various changes can be made therein without departing from the spirit and scope of the invention.

1-63. (canceled)

64. A method of growing an algal culture having a fatty acid content of at least 5.0×10̂-12 grams, the method comprising:
(a) adding an algal culture to a growth medium including water, an alkaline buffer solution, phosphate, and nitrogen; and
(b) exposing the algal culture to a light condition greater than about 60 μE/m²/sec, wherein the light schedule includes at least 6 hours of light followed by at least 6 hours of darkness.

65. A growth medium for an alga, comprising
(a) water;
(b) an alkaline buffer solution;
(c) a trace metal ion solution;
(d) a vitamin solution;
(e) phosphate; and
(f) nitrogen.

66. A method of selectively generating an algal culture having an identification property, the method comprising:
(a) obtaining a first algal culture having an identification property having a first value;
(b) isolating the first algal culture in a first growth medium;
(c) incubating the first algal culture in the first growth medium to provide a second algal culture;
(d) sorting the second algal culture to select algal cells having the identification property having a second value to provide a sorted portion of the second algal culture.

67. The method of claim 66, wherein the identification property is selected from the group consisting of high lipid content, high biomass content, rapid growth rate, fatty acid profile, and combinations thereof.

68. The method of claim 66, wherein the first algal culture is a strain variant of Chrysochromulina sp.

69. The method of claim 66, wherein the second algal culture is a strain variant of Chrysochromulina sp. different from the first algal culture.

70. The method of claim 66, wherein the second value is improved over the first value.

71. The method of claim 66, wherein the second algal culture is sorted using flow cytometry and a lipophilic dye.

72. The method of claim 71, wherein the lipophilic dye is 4,4-difluoro-1,3,5,7-tetramethyl-4-bora-3a,4a-diaza-s-indacene.

73. The method of claim 66, wherein the first growth medium is the same as the second growth medium.

74. The method of claim 66, wherein the first growth medium is different from the second growth medium.

75. The method of claim 66, further comprising isolating the sorted portion of the second algal culture in a second growth medium, incubating the sorted portion of the second algal culture in the second growth medium to provide a third algal culture, and sorting the third algal culture to select algal cells having the identification property having a third value to provide a sorted portion of the third algal culture.

76. The method of claim 75, wherein the second growth medium is the same as the third growth medium.

77. The method of claim 75, wherein the second growth medium is different from the third growth medium.

78. The method of claim 66, wherein the identification property is lipid content and wherein the second value is at least 5% greater than the first value.

79. The method of claim 75, wherein the identification property is lipid content and wherein the third value is at least 5% greater than the first value.

80. The method of claim 66, wherein the identification property is lipid content and the identification value has an improved second value compared to the first value selected from the group consisting of at least 15% improvement, at least 20% improvement, and at least 25% improvement.

81. The method of claim 66, wherein the identification property is saturate content and the identification value has an improved second value compared to the first value selected from the group consisting of at least 15% improvement, at least 20% improvement, and at least 25% improvement.

82. A fatty acid mixture obtained from an alga, comprising:
(a) C14 in an amount in the range of about 14 to about 25 weight percent of the total lipid content;
(b) C16 in an amount in the range of about 17 to about 26 weight percent of the total lipid content;
(c) C18 in an amount in the range of about 29 to about 57 weight percent of the total lipid content; and
(d) C20 and greater in an amount in the range of about 9 to about 30 weight percent of the total lipid content.

83. An algal culture including a plurality of algal cells having an average fatty acid content of at least 5.0×10̂-12 grams per cell, wherein the algal culture is capable of surviving in fresh water.
